(19) United States
(12) Patent Application Publication
(10) Pub. No.: US 2002/0151707 A1
(43) Pub. Date: Oct. 17, 2002

(54) Immune mediators and related methods

(75) Inventors: Wayne Kindsvogel, Seattle, WA (US); Eva Pia Reich, Palo Alto, CA (US); Jane A. Gross, Seattle, WA (US); Shrinkant Deshpande, Fremont, CA (US); Paul O. Sheppard, Redmond, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corixa Corp., Seattle, WA (US)

(21) Appl. No.: 10/081,281

(22) Filed: Feb. 20, 2002

Related U.S. Application Data
(63) Continuation of application No. 09/261,811, filed on Mar. 3, 1999.
Application No. 09/261,811 is a continuation of application No. 08/657,581, filed on Jun. 7, 1996, now abandoned.
Application No. 09/261,811 is a continuation of application No. 08/480,002, filed on Jun. 7, 1995, now abandoned.
Application No. 09/261,811 is a continuation of application No. 08/483,241, filed on Jun. 7, 1995, now abandoned.
Application No. 09/261,811 is a continuation of application No. 08/482,133, filed on Jun. 7, 1995, now abandoned.
(60) Provisional application No. 60/005,964, filed on Oct. 27, 1995.

(51) Int. Cl. C07K 14/74; C12P 21/02; C12N 5/06; C07H 21/04

(52) U.S. Cl. 536/23.5; 530/350; 435/69.1; 435/320.1; 435/325

(57) ABSTRACT

Immune modulators, such as soluble, fused MHC heterodimers and soluble, fused MHC heterodimer:peptide complexes, are described. Related methods and peptides are also disclosed. In a preferred aspect, these mediators and methods are related to autoimmunity.

### RELATED CASES

The present application is a continuation-in-part of U.S. Ser. No. 08,/480,002, filed Jun. 7, 1995, U.S. Ser. No. 08/483,241, filed Jun. 7, 1995 and U.S. Ser. No. 08/482,133, filed Jun. 7, 1995, and claims the benefit of U.S. Provisional Application No. 60/005,964, filed Oct. 27, 1995 which applications are pending.

### BACKGROUND OF THE INVENTION

There is currently a great interest in developing pharmaceuticals based on the growing understanding of the structure and function of the major histocompatibility complex (MHC) antigens. These cell surface glycoproteins are known to play an important role in antigen presentation and in eliciting a variety of T cell responses to antigens.

T cells, unlike B cells, do not directly recognize antigens. Instead, an accessory cell must first process an antigen and present it in association with an MHC molecule in order to elicit a T cell-mediated immunological response. The major function of MHC glycoproteins appears to be the binding and presentation of processed antigen in the form of short antigenic peptides.

In addition to binding foreign or “non-self” antigenic peptides, MHC molecules can also bind “self” peptides. If T lymphocytes then respond to cells presenting “self” or autoantigenic peptides, a condition of autoimmunity results. Over 30 autoimmune diseases are presently known, including myasthenia gravis (MG), multiple sclerosis (MS), systemic lupus erythematosus (SLE), rheumatoid arthritis (RA), insulin-dependent diabetes mellitus (IDDM), etc. Characteristic of these diseases is an attack by the immune system on the tissues of the host. In non-diseased individuals, such attack does not occur because the immune system recognizes these tissues as “self ”. Autoimmunity occurs when a specific adaptive immune response is mounted against self tissue antigens.

Insulin-dependent diabetes mellitus (IDDM), also known as Type I diabetes, results from the autoimmune destruction of the insulin-producing β-cells of the pancreas. Studies directed at identifying the autoantigen(s) responsible for β-cell destruction have identified several candidates, including insulin (Palmer et al., Science 222: 1337-1339, 1983), a poorly characterized islet cell antigen (Bottazzo et al., Lancet ii: 1279-1283, 1974), and a 64 kDa antigen that has been shown to be glutamic acid decarboxylase (Baekkeskov et al., Nature 298: 167-169 (1982); Baekkeskov et al., Nature 347: 151-156, 1990). Antibodies to glutamic acid decarboxylase (hereinafter referred to as “GAD”) have been found to be present in patients prior to clinical manifestation of IDDM (Baekkeskov et al, J. Clin. Invest. 79: 926-934, 1987).

GAD catalyzes the rate-limiting step in the synthesis of γ-aminobutyric acid (GABA), a major inhibitory neurotransmitter of the mammalian central nervous system. Little is known with certainty regarding the regulation of GAD activity or the expression of GAD genes. Despite its wide distribution in the brain, GAD protein is present in very small quantities and is very difficult to purify to homogeneity. GAD has multiple isoforms encoded by different genes. These multiple forms of the enzyme differ in molecular weight, kinetic properties, sequence (when known), and hydrophobic properties. For example, the presence of three different forms of GAD in porcine brain has been reported (Spink et al., J. Neurochem. 40:1113-1119, 1983), as well as four forms in rat brain (Spink et al., Brain Res. 421:235-244, 1987). A mouse brain GAD (Huang et al., Proc. Natl. Acad. Sci. USA 87:8491-8495, 1990) and a GAD clone isolated from feline brain (Kobayashi et al., J. Neurosci. 2:2768-2772, 1987) have also been reported. At least two isomers of GAD have been reported in human brain (Chang and Gottlieb, J. Neurosci. 8:2123-2130, 1988). A human pancreatic islet cell GAD has recently been characterized by molecular cloning (Lernmark et al., U.S. patent application 07/702,162; PCT publication WO 92/20811). This form of GAD is identical to one subsequently identified human brain isoform (Bu et al., Proc. Natl. Acad. Sci. USA 89:2115-2119, 1992). A second GAD isoform identified in human brain is not present in human islets (Karlsen et al., Diabetes 41:1355-1359, 1992).

It has been suggested that the inflammatory CD4⁺(TH¹) T cell response to GAD is the primary autoantigen reactivity, arising at the same time as the onset of insulitis in NOD mice, followed subsequently by T-cell reactivity to other β-cell antigens. At the same time, the initial T-cell response to GAD has been reported to be limited to one region of the GAD polypeptide, with spread to additional GAD determinants over time (WO 95/07992; Kaufman et al., Nature 366: 69-71, 1993; and Tisch et al., Nature 366: 72-75, 1993).

Evidence suggests that GAD is the primary autoantigen responsible for initiating the β cell assault leading to diabetes both in humans and in animal models. Three peptides derived from mouse and human GAD65, peptide #17 sequence 246-266, peptide #34 sequence 509-528 and peptide #35 sequence 524-543, have been implicated as candidates for the autoantigen by their ability to induce a T cell response in mice (Kaufman et al., ibid)

Current treatment for autoimmune disease and related conditions consists primarily of treating the symptoms, but not intervening in the etiology of the disease. Broad spectrum chemotherapeutic agents are typically employed, which agents are often associated with numerous undesirable side effects. Therefore, there is a need for compounds capable of selectively suppressing autoimmune responses by blocking MHC binding, thereby providing a safer, more effective treatment. In addition, such selective immunosuppressive compounds are needed in the treatment of non-autoimmune diseases, such as graft versus-host disease (GVHD) or various allergic responses. For instance, chronic GVHD patients frequently present conditions and symptoms similar to certain autoimmune diseases.

The inadequate autoimmune disease treatments presently available illustrate the urgent need to identify new agents that block MHC-restricted immune responses, but avoid undesirable side effects, such as nonspecific suppression of an individual's overall immune response. A desirable approach to treating autoimmune diseases and other pathological conditions mediated by MHC would be to use soluble, fused MHC heterodimer:peptide complexes to acheive immune tolerence or anergy to T cells which respond to antigenic peptides. The present invention fulfills such needs, and provides related advantages.

Identification of synthetic antigenic peptides, and demonstration that these peptides bind selectively to MHC molecules associated with disease and that stimulates T cells would help to implicate a particular peptide or peptide:MHC complex in susceptibility to an autoimmune disease. The present invention fulfills such needs, and provides related advantages.

### SUMMARY OF THE INVENTION

Within a first aspect the present invention provides a soluble, fused MHC heterodimer:peptide complex comprising a first DNA segment encoding at least a portion of a first domain of a selected MHC molecule; a second DNA segment encoding at least a portion of a second domain of the selected MHC molecule; a first linker DNA segment encoding about 5 to about 25 amino acids and connecting in-frame the first and second DNA segments; wherein linkage of the first DNA segment to the second DNA segment by the first linker DNA segment results in a fused first DNA-first linker-second DNA polysegment; a third DNA segment encoding an antigenic peptide capable of associating with a peptide binding groove of the selected MHC molecule a second linker DNA segment encoding about 5 to about 25 amino acids and connecting in-frame the third DNA segment to the fused first DNA-first linker-second DNA polysegment wherein linkage of the third DNA segment to the fused first DNA-first linker-second DNA polysegment by the second linker DNA segment results in a soluble, fused MHC heterodimer:peptide complex.

Within one embodiment the selected MHC molecule is an MHC Class II molecule.

Within another embodiment the first DNA segment encodes a β1 domain.

Within yet another embodiment the second DNA segment encodes an al domain or α1α2 domains.

Within another embodiment the selected MHC molecule is selected from the group consisting of IAg^{g7}, IA^{s}, DR1β*1501 and DRA*0101.

Within a further embodiment the selected MHC molecule is an MHC Class I molecule.

Within still another embodiment the first linker DNA segment is GASAG (SEQ. ID. NO. 29) or GGGGSGGGGSGGGGS (SEQ. ID. NO. 36).

Within yet another embodiment the second linker DNA segment is GGSGG (SEQ. ID. NO. 30) or GGGSGGS (SEQ. ID. NO. 31).

Within a further embodiment the third DNA segment encodes an antigenic peptide capable of stimulating an MHC-mediated immune response.

Within another embodiment the peptide is selected from the group consisting of a mammalian GAD 65 peptide, (SEQ ID NO: 59), (SEQ. ID. NO. 61), (SEQ ID NO:40), (SEQ. ID. NO. 39) and a mammalian mylein basic peptide(SEQ. ID. NO. 33).

The invention further provides the soluble, fused MHC heterodimer:peptide complex, wherein said MHC heterodimer:peptide complex further comprises a fourth DNA segment encoding at least a portion of a third domain of the selected MHC molecule, and a third linker DNA segment encoding about 5 to about 25 amino acids and connecting in-frame the second and fourth DNA segments resulting in a fused third DNA-second linker-first DNA-first linker-second DNA-third linker-fourth DNA polysegment.

Within one embodiment the selected MHC molecule is an MHC Class I molecule.

Within a second embodiment the selected MHC molecule is an MHC Class II molecule.

Within another embodiment the fourth DNA segment is a β2 chain.

Within yet another embodiment the third linker DNA segment is GGGGSGGGGSGGGGSGGGGSGGGGS (SEQ. ID. NO. 32).

Within a second aspect, the invention provides an isolated polynucleotide molecule encoding a soluble, fused MHC heterodimer:peptide complex.

Within a third aspect, the invention further provides a fusion protein expression vector capable of expressing a soluble, fused MHC heterodimer:peptide complex, comprising the following operably linked elements, a transcription promoter; a first DNA segment encoding at least a portion of a first domain of a selected MHC molecule; a second DNA segment encoding at least a portion of a second domain of the selected MHC molecule; a first linker DNA segment encoding about 5 to about 25 amino acids and connecting in-frame the first and second DNA segments; wherein linkage of the first DNA segment to the second DNA segment by the first linker DNA segment results in a fused first DNA-first linker-second DNA polysegment; a third DNA segment encoding an antigenic peptide capable of associating with a peptide binding groove of the selected MHC molecule; a second linker DNA segment encoding about 5 to about 25 amino acids and connecting in-frame the third DNA segment to the fused first DNA-first linker-second DNA polysegment; wherein linkage of the third DNA segment to the fused first DNA-first linker-second DNA polysegment by the second linker DNA segment results in expression of a soluble, fused MHC heterodimer:peptide complex; and a transcription terminator.

Within one embodiment the invention provides the expression vector, wherein the MHC heterodimer:peptide complex further comprises a fourth DNA segment encoding at least a portion of a third domain of the selected MHC molecule, and a third linker DNA segment encoding about 5 to about 25 amino acids and connecting in-frame the second and fourth DNA segments resulting in a fused third DNA-second linker-first DNA-first linker-second DNA-third linker-fourth DNA polysegment.

Within a another aspect, the invention provides a soluble, fused MHC heterodimer:peptide complex produced by culturing a cell into which has been introduced an expression vector, whereby said cell expresses a soluble, fused MHC heterodimer:peptide complex encoded by the DNA polysegment; and recovering the soluble, fused MHC heterodimer:peptide complex.

Within yet another apsect the invention provides a pharmaceutical composition comprising a soluble, fused MHC heterodimer:peptide complex in combination with a pharmaceutically acceptable vehicle.

Within another aspect the invention provides an antibody that binds to an epitope of a soluble, fused MHC heterodimer:peptide complex.

Within yet another aspect the invention provides a method of treating a patient to decrease an autoimmune response, the method comprising inducing immunological tolerance in said patient by administering a therapeutically effective amount of a soluble, fused MHC heterodimer:peptide complex of claim 1.

Within still another aspect the invention provides a method for preparing a responder cell clone that proliferates when combined with a selected antigenic peptide presented by a stimulator cell, comprising isolating non-adherent, CD56-, CD8-cells that are reactive with the selected antigenic peptide, thereby forming responder cells; stimulating the responder cells with pulsed or primed stimulator cells; restimulating the stimulated responder cells with pulsed or primed stimulator cells; and isolating a responder cell clone.

Within one embodiment the responder cells are isolated from a prediabetic or new onset diabetic patient.

Within a second embodiment the responder cell clone is a T cell clone.

Within another aspect the selected antigenic peptide is a GAD peptide.

These and other aspects of the invention will become evident upon reference to the following detailed description.

### DETAILED DESCRIPTION OF THE INVENTION

Prior to setting forth the invention, it may be helpful to an understanding thereof to provide definitions of certain terms to be used hereinafter:

Fused MHC Heterodimer:Peptide Complex:

As used herein it refers to a fusion protein such as the fused, MHC heterodimer:peptide complex of the invention. Such fusion proteins will be indicated with a colon(:). MHC-peptide complexes which are not fusion proteins, are native MHC containing protein or exogenously loaded MHC molecules are indicated with a dash (-).

A Domain of a Selected MHC Molecule:

A portion of an MHC domain which is sufficient to form, either alone, or in combination with another portion of an MHC domain, a peptide binding site which is capable of presenting an antigentic peptide in such a fashion that it is recognized by a T cell receptor. Such MHC domains would include the extracellular portion of the two polypeptide chains of either Class I or Class II MHC. This would include any or all of the domains of α chain (α1,α2, or (α3) and β2-microgloublin subunit of Class I MHC. For example, Class I MHC domains would include any combination of the three a chain domains either independent of the others, α1, α2, or α3, in tandem, α1α2, α2α3, α1α3) and and/or the β2 domain. Also included are the a chain (a1, a2) and P chain (β1,β2) of Class II MHC. This would include α1 or β2 independent of the other, or β1 and α2 in tandem (α1α2). It would also include β1 or β2 independent of the other, or β1 and β2 in tandem (β1β2).

Linker DNA Segment

A segment of DNA encoding about 5 to about 25 amino acids, prototypically repeating glycine residues with interspersed serine residues which forms a flexible link between two DNA segments. This flexible link allows the two DNA segments to attain a proper configuration, such as an MHC peptide binding groove, or allows a peptide to properly bind into such a groove.

Antigenic Peptide:

A peptide which contains an epitope recognized by immune cells, particularly T cells, and is capable of stimulating an MHC-mediated immune response.

The major histocompatibility complex (MHC) is a family of highly polymorphic proteins, divided into two classes, Class I and Class II, which are membrane-associated and present antigen to T lymphocytes (T cells). MHC Class I and Class II molecules are distinguished by the types of cells on which they are expressed, and by the subsets of T cells which recognize them. Class I MHC molecules (e.g., HLA-A, -B and -C molecules in the human system) are expressed on almost all nucleated cells and are recognized by cytotoxic T lymphocytes (CTL), which then destroy the antigen-bearing cells. Class II MHC molecules (HLA-DP, -DQ and -DR, for example, in humans) are expressed primarily on the surface of antigen-presenting cells, such as B lymphocytes, dendritic cells, macrophages, and the like. Class II MHC is recognized by CD4⁺T helper lymphocytes (T_{H}) T_{H} cells induce proliferation of both B and T lymphocytes, thus amplifying the immune response to the particular antigenic peptide that is displayed (Takahashi, Microbiol. Immunol., 37:1-9, 1993). Two distinct antigen processing pathways are associated with the two MHC classes. Intracellular antigens, synthesized inside of the cell, such as from viral or newly synthesized cellular proteins, for example, are processed and presented by Class I MHC. Exogenous antigens, taken up by the antigen-presenting cell (APC) from outside of the cell through endocytosis, are processed and presented by Class II MHC. After the antigenic material is proteolytically processed by the MHC-bearing cell, the resulting antigenic peptide forms a complex with the antigen binding groove of the MHC molecule through various noncovalent associations. The MHC-peptide complex on the cell surface is recognized by a specific T cell receptor on a cytotoxic or helper T cell.

The MHC of humans (also referred to as human leukocyte antigens (HLA)) on chromosome 6 has three loci, HLA-A, HLA-B and HLA-C, the first two of which have a large number of alleles encoding alloantigens. An adjacent region, known as HLA-D, is subdivided into HLA-DR, HLA-DQ and HLA-DP. The HLA region is now known as the human MHC region, and is equivalent to the H-2 region in mice. HLA-A, -B and -C resemble mouse H-2K, -D, and -L and are the Class I MHC molecules. HLA-DP, -DQ and -DR resemble mouse I-A and I-E and are the Class II molecules. MHC glycoproteins of both classes have been isolated and characterized (see Fundamental Immunology, 2d Ed., W. E. Paul (ed.), Ravens Press, N.Y. (1989); and Roitt et al., Immunology, 2d Ed., Gower Medical Publishing, London (1989), which are both incorporated herein by reference).

Human MHC Class I molecules consist of a polymorphic type I integral membrane glycoprotein heavy chain of about 46 kD, noncovalently associated with a 12 kD soluble subunit, β2-microglobulin. The heavy chain consists of two distinct extracellular regions, the membrane distal, peptide binding region formed by the al and α2 domains, and the membrane proximal, CD8-binding region derived from the α3 domain. β₂-microglobulin is a single, compact immunogobulin-like domain that lacks a membrane anchor, and exists either associated with the class I heavy chain or free in plasma (Germain and Margulies, Annu. Rev. Immunol. 11:403-50, 1993).

Human MHC Class II is a heterodimeric integral membrane protein. Each dimer consists of one a and one chain in noncovalent association. The two chains are similar to each other, with the a chain having a molecular weight of 32-34 kD and the β chain having a molecular weight of 29-32 kD. Both polypeptide chains contain N-linked oligosaccharide groups and have extracellular amino termini and intracellular carboxy termini.

The extracellular portions of the α and β chain that comprise the class II molecule have been subdivided into two domains of about 90 amino acids each, called 60 1, α2, and β1, β2, respectively. The α2 and β2 domains each contain a disulfide-linked loop. The peptide-binding region of the class II molecule is -formed by the interaction of the α1 and β1 domains. This interaction results in an open-ended, antigenic peptide-binding groove made up of two a helices, and an eight-stranded β-pleated sheet platform.

The α and β chains of Class II molecules are encoded by different MHC genes and are polymorphic (see Addas et al., Cellular and Molecular Immunology, 2d Ed., W. B. Saunders Co., New York (1994), which is incorporated by reference in its entirety). Within the present invention, a preferred a chain is DRA*0101 and a preferred β chain is DRβ1*1501.

The immunological properties of MHC histocompatibility proteins are largely defined by the antigenic peptide that is bound to them. An antigenic peptide is one which contains an amino acid sequence recognized by immune cells, e.g., T cells. Antigenic peptides for a number of autoimmune diseases are known. For example, in experimentally induced autoimmune diseases, antigens involved in pathogenesis have been characterized: in arthritis in rat and mouse, native type II collagen is identified in collagen-induced arthritis, and mycobacterial heat shock protein in adjuvant arthritis (Stuart et al., Ann. Rev. Immunol. 2:199-218, 1984; and van Eden et al., Nature 331:171-173, 1988); thyroglobulin has been identified in experimental allergic thyroiditis (EAT) in mice (Marion et al., J. Exp. Med. 152:1115-1120, 1988); acetyl-choline receptor (AChR) in experimental allergic myasthenia gravis (EAMG) (Lindstrom et al., Adv. Immunol. 42:233-284, 1988); and myelin basic protein (MBP) and proteolipid protein (PLP) in experimental allergic encephalomyelitis (EAE) in mouse and rat (Acha-Orbea et al., Ann. Rev. Imm. 7:377-405, 1989). In addition, target antigens have been identified in humans: type II collagen in human rheumatoid arthritis (Holoshitz et al., Lancet ii:305-309, 1986) and acetylcholine receptor in myasthenia gravis (Lindstrom et al., Adv. Immunol. 42:233-284, 1988).

Soluble, fused MHC heterodimer:peptide complexes of the present invention can be used as antagonists to therapeutically block the binding of particular T cells and antigen-presenting cells. In addition, the molecules can induce anergy, or proliferative nonreponsiveness, in targeted T cells. A soluble, fused MHC heterodimer:peptide molecule directed toward a desired autoimmune disease contains the antigenic peptide implicated for that autoimmune disease properly positioned in the binding groove of the MHC molecule, without need for solublization of MHC or exogenous loading of an independently manufactured peptide.

Previous methods for producing desirable MHC Class II histocompatibility proteins have provided material that contains a mixture of antigenic peptides (Buus et al., Science 242:1045-1047, 1988; and Rudensky et al., Nature 353:622-627, 1991), which can be only partially loaded with a defined antigenic peptide (Watts and McConnel, Proc. Natl. Acad. Sci. USA 83:9660-64, 1986; and Ceppellini et al., Nature 339:392-94, 1989). Various methods have been developed to produce heterodimers that do not present endogenous antigens (Stern and Wiley, Cell 68:465-77, 1992; Ljunggren et al., Nature 346:476-80, 1990; and Schumacher et al., Cell 62:563-67, 1990) that can be loaded with a peptide of choice. WO 95/23814 and Kozono et al. have described production of soluble murine Class II molecules, I-E^{dk} and I-A^{d}, each with a peptide attached by a linker to the N terminus of the β chain. Ignatowicz et al. (J. Immunol. 154:38-62, 1995) have expressed membrane-bound I-A^{d} with peptide attached. These methods incorporate the use of both membrane-bound heterodimer and soluble heterodimer.

The current invention offers the advantage of a soluble, fused MHC heterodimer made up of two or more MHC domains joined together via a flexible linkage, and onto which is tethered (via an additional flexible linkage) an antigenic peptide which is able to bind to the peptide binding groove presented by the soluble, fused MHC heterodimer. Such a complex provides an MHC molecule which is soluble and, because the components of the heterodimer and corresponding antigenic peptide are permanently linked into a single chain configuration, there is no need for complex heterodimer truncation or formation. These complexes eliminate inefficient and nonspecific peptide loading. Producing the claimed MHC:peptide complexes by recombinant methodology results in specific, high yield protein production, where the final product contains only the properly configured MHC:peptide complex of choice. As used herein, a soluble heterodimer is one that does not contain membrane-associated MHC. The soluble MHC heterodimer of the present invention has never been membrane-associated. Further, the polypeptides contained within the MHC heterodimer do not contain an amino acid sequence capable of acting as a transmembrane domain or as a cytoplasmic domain.

The present invention provides a soluble, fused MHC heterodimer which contains an antigenic peptide covalently attached to the amino terminal portion of an a or β chain of MHC through a peptide linkage, and the C terminal of the linked α or β chain may be attached to the N terminal portion of another α or β chain, there by creating a two, or three domain MHC molecule. The invention further provides a linkage connecting an additional domain to provide a four domain MHC molecule. The a chain portion can include: α1 or α2 independent of the other or α1 and α2 in tandem (α1α2), or joined together through an intervening peptide linkage. The β chain portion can include, β1 or β2 independent, β1β2, β1 and β2 in tandem, or joined together through an intervening peptide linkage. Combinations of α1, α2, β1 and β2 can also be created through flexible linkers, such as β1α1, or β1α1α2, for example.

The soluble, fused MHC heterodimer:peptide complexes of the present invention comprise a first DNA segment encoding at least a portion of a first domain of a selected MHC molecule; a second DNA segment encoding at least a portion of a second domain of the selected MHC molecule; a first linker DNA segment encoding about 5 to about 25 amino acids and connecting in-frame the first and second DNA segments; wherein linkage of the first DNA segment to the second DNA segment results in a fused first DNA-first linker-second DNA polysegment; a third DNA segment encoding an antigenic peptide capable of associating with a peptide binding groove of the selected MHC molecule; a second linker DNA segment encoding about 5 to about 25 amino acids and connecting in-frame the third DNA segment to the fused first DNA-first linker-second DNA polysegment wherein linkage of the third DNA segment to the fused first DNA-first linker-second DNA polysegment by the second linker DNA segment results in a soluble, fused MHC heterodimer:peptide complex. The invention also provides soluble, fused MHC heterodimer:peptide complexes which contain a fourth DNA segment encoding at least a portion of a third domain of a selected MHC molecule and a third linker DNA segment encoding about 5 to about 25 amino acids and connecting in-frame the second and fourth DNA segments resulting in a fused third DNA-first linker-first DNA-second linker-second DNA-third linker-fourth DNA polysegment.

The first, second, third and fourth DNA segments Of a selected MHC molecule may contain a portion of the heavy chain or β₂-microgloublin subunit of Class I MHC. This would include portions of any combination of the three extracellular domains (α1, α2, α3, α1α2, or α2α3) as well as the β₂ domain. This also includes the α chain or β chain of a Class II MHC molecule. This would include portions of al or α2 independent of the other or α1 and α2 in tandem (α1α2). It would also include portions of β1 or β2 independent, β1 and β2 in tandem (β1β2). The soluble, fused MHC heterodimer:peptide complexes of the invention can be represented by combinations of α1, α2, β1 and β2 created through flexible linkers, such as peptide-β1α1, peptide-β1α1α2, or peptide-β1α1α2β2, for example.

Linkers of the current invention may be from about 5 to about 25 amino acids in length, depending on the molecular model of the MHC or MHC:peptide complex. Preferably, flexible linkers are made of repeating Gly residues separated by one or more Ser residues to permit a random, flexible motion. In the case of Class II MHC complexes this flexibility accommodates positioning of the α and β segments to properly configure the binding groove, and also allows for maximum positioning of the peptide in the groove. Linker position and length can be modeled based on the crystal structure of MHC Class II molecules (Brown et al., Nature 364:33-39, 1993), where α1 and β1 are assembled to form the peptide binding groove. Linkers joining segments of the α and β chains together are based on the geometry of the region in the hypothetical binding site and the distance between the C terminus and the N terminus of the relevant segments. Molecular modeling based on the X-ray crystal structure of Class II MHC (Stern et al., Nature 368:215-221, 1994) dictates the length of linkers joining antigenic peptide, α chain segments and β chain segments.

The soluble, fused heterodimer MHC:peptide complexes of the present invention can incorporate cDNA from any allele that predisposes or increased the likelyhood of susceptibility to a specific autoimmune disease. Specific autoimmune diseases are correlated with specific MHC types. Specific haplotypes have been associated with many of the autoimmune diseases. For example, HLA-DR2⁺ and HLA-DR3⁺ individuals are at a higher risk than the general population to develop systemic lupus erythematosus (SLE) (Reinertsen et al., N. Engl. J. Med. 299:515-18, 1970). Myasthenia gravis has been linked to HLA-D (Safwenberg et al., Tissue Antigens 12:136-42,1978. Susceptibility to rheumatoid arthritis is associated with HLA-D/DR in humans. Methods for identifying which alleles, and subsequently which MHC-encoded pblypeptides, are associated with an autoimmune disease are known in the art. Exemplary alleles for IDDM include DR4, DQ8, DR3, DQ3.2.

The amino acid sequence of each of a number of Class I and Class II proteins are known, and the genes or cDNAs have been cloned. Thus, these nucleic acids can be used to express MHC polypeptides. If a desired MHC gene or cDNA is not available, cloning methods known to those skilled in the art may be used to isolate the genes. One such method that can be used is to purify the desired MHC polypeptide, obtain a partial amino acid sequence, synthesize a nucleotide probe based on the amino acid sequence, and use the probe to identify clones that harbor the desired gene from a cDNA or genomic library.

The invention also provides methods for preparing responder T-cell clones that proliferate when combined with a selected antigenic peptide presented by a stimulator cell. Such clones can be used to identify and map antigenic peptides associated with autoimmune disease. These peptides can then be incorporated into the soluble, fused MHC heterodimer:peptide complexes of the invention. The method provides isolation and enrichment of non-adherent, CD56⁻, CD8⁻ T cells that are reactive with a selected antigenic peptide. These cells are herein referred to as responder cells. Suitable responder cells can be isolated, for example, from peripheral blood mononuclear cells (PBMNC) obtained from patients prior to or after onset of an autoimmune disease of interest. For example, PBMNCs can be obtained from prediabetic and new onset diabetic patients. These patients can be prescreened for specific HLA markers, such as DR3-DR4 or DQ3.2, which have the highest association with susceptibility to IDDM. From the collected PBMNCs, a portion is kept to serve as stimulator cells. From the remainder, the desired autoreactive responder cells are purified and isolated by two rounds of plating, to remove adherent cells from the population, followed by removal of monocytes and B cells with nylon wool. Enrichment for non-adherent CD4+T cells is completed by sequential plating of the cells onto plates coated with anti-CD8 and anti-CD56 antibodies.

The stimulator cells are pulsed or primed with whole GAD or an appropriate antigenic peptide. For example, stimulator cells from the PBMNCs of IDDM patients can be stimulated with antigenic GAD peptides then combined with PBMNCs or responder cells. After seven or 14 days, responder cell (T cell) clones are generated through limiting dilution and tested for antigen reactivity.

These responder cell (T cell) clones can then be used, for example, to map epitopes which bind to MHC and are recognized by a particular T cell. One such method uses overlapping peptide fragments of the autoantigen which are generated by tryptic digestion, or more preferably, overlapping peptides are synthesized using known peptide synthesis techniques. The peptide fragments are then tested for their ability to stimulate the responder T cell clones or lines (see, for example, Ota et al., Nature, 346:183-187, 1990).

Once such a peptide fragment has been identified, synthetic antigenic peptides can be specifically designed, for example, to enhance the binding affinity for MHC and to out-compete any naturally processed peptides. Such synthetic peptides, when combined into a soluble, fused MHC heterodimer:peptide complex, would allow manipulation of the immune system in vivo, in order to tolerize or anergize disease-associated activated T cells, thereby ameliorating the autoimmune disease.

Dissecting the functional role of individual peptides and peptide clusters in the interaction of a peptide ligand with an MHC molecule, and also in subsequent T cell recognition and reactivity, is a difficult undertaking due to the degeneracy of peptide binding to the MHC. Changes in T cell recognition or in the ability of an altered peptide to associate with MHC can be used to establish that a particular amino acid or group of amino acids comprises part of an MHC or T cell determinant. The interactions of altered peptides can be further assessed by competition with the parental peptide for presentation to a T cell, or through development of direct peptide-MHC binding assays. Changes to a peptide that do not involve MHC binding could well affect T cell recognition. For example, in a peptide, specific MHC contact points might only occur within a central core of a few consecutive or individual amino acids, whereas those amino acids involved in T cell recognition may include a completely different subset of residues.

In a preferred method, residues that alter T cell recognition are determined by substituting amino acids for each position in the peptide in question, and by assessing whether such change in residues alters the peptide's ability to associate with MHC (Allen et al., Nature 327:713-15, 1987; Sette et al., Nature 328:395-99, 1987; O'Sullivan et al., J. Immunol. 147:2663-69, 1991; Evavold et al., J. Immunol. 148:347-53, 1992; Jorgensen et al., Annu. Rev. Immunol. 10:835-73, 1992; Hammer et al., Cell 74:197-203, 1993; Evavold et al., Immunol. Today 14:602-9, 1993; Hammer et al., Proc. Natl. Acad. Sci. USA 91:4456-60, 1994; and Reich et al., J. Immunol. 154:2279-88, 1994). One method would involve generating a panel of altered peptides wherein individual or groups of amino acid residues are substituted with conservative, semi-conservative or non-conservative residues. A preferred variant of this method is an alanine scan (Ala scan) where a series of synthetic peptides are synthesized wherein each individual amino acid is substituted with L-alanine (L-Ala scan). Alanine is the amino acid of choice because it is found in all positions (buried and exposed), in secondary structure, it does not impose steric hindrances, or add additional hydrogen bonds or hydrophobic side chains. Alanine substitutions can be done independently or in clusters depending on the information desired. Where the information pertains to specific residues involved in binding, each residue in the peptide -under investigation can be converted to alanine and the binding affinity compared to the unsubstituted peptide. Additional structural and conformational information regarding each residue and the peptide as a whole can be gained, for example, by synthesizing a series of analogs wherein each residue is substituted with a D-amino acid such as D-alanine (D-Ala scan) (Galantino et al., in Smith, J. and Rivier, J. (eds.), Peptides Chemistry and Biology (Proceedings of the Twelfth American Peptide Symposium), ESCOM, Leiden, 1992, pp. 404-05). Essential residues can be identified, and nonessential residues targeted for modification, deletion or replacement by other residues that may enhance a desired quality (Cunningham and Wells, Science, 244:1081-1085, 1989; Cunningham and Wells, Proc. Natl. Acad. Sci. USA, 88:3407-3411, 1991; Ehrlich et al., J. Biol. Chem. 267:11606-11, 1992; Zhang et al., Proc. Natl. Acad. Sci. USA 90:4446-50, 1993; see also “Molecular Design and Modeling: Concepts and Applications Part A Proteins, Peptides, and Enzymes,” Methods in Enzymology, Vol. 202, Langone (ed.), Academic Press, San Diego, Calif., 1991).

Truncated peptides can be generated from the altered or unaltered peptides by synthesizing peptides wherein amino acid residues are truncated from the N- or C-terminus to determine the shortest active peptide, or between the N- and C-terminus to determine the shortest active sequence. Such peptides could be specifically developed to stimulate a response when joined to a particular MHC to form a peptide ligand to induce anergy in appropriate T cells in vivo or in vitro.

The physical and biological properties of the soluble, fused MHC heterodimer:peptide complexes may be assessed in a number of ways. Mass spectral analysis methods such as electrospray and Matrix-Assisted Laser Desorption/Ionization Time Of Flight mass spectrometry (MALDI TOF) analysis are routinely used in the art to provide such information as molecular weight and confirm disulfide bond formation. FACs analysis can be used to determine proper folding of the single chain complex.

An ELISA (Enzyme-linked Immunosorbent Assay) can be used to measure concentration and confirm correct folding of the soluble, fused MHC heterodimer:peptide complexes. This assay can be used with either whole cells; solublized MHC, removed from the cell surface; or free soluble, fused MHC heterodimer:peptide complexes of the current invention. In an exemplary ELISA, an antibody that detects the recombinant MHC haplotype is coated onto wells of a microtiter plate. In a preferred embodiment, the antibody is L243, a monoclonal antibody that recognizes only correctly folded HLA-DR MHC dimers. One of skill in the art will recognize that other MHC Class II-specific antibodies are known and available. Alternatively, there are numerous routine techniques and methodologies in the field for producing antibodies (for example, Hurrell, J. G. R. (ed)., Monoclonal Hybridoma Antibodies: Techniques and Applications, CRC Press Inc., Boca Raton, Fla., 1982), if an appropriate antibody for a particular haplotype does not exist. Anti-MHC Class II antibodies can also be used to purify Class II molecules through techniques such as affinity chromatography, or as a marker reagent to detect the presence of Class II molecules on cells or in solution. Such antibodies are also useful for Western analysis or immunoblotting, particularly of purified cell-secreted material. Polyclonal, affinity purified polyclonal, monoclonal and single chain antibodies are suitable for use in this regard. In addition, proteolytic and recombinant fragments and epitope binding domains can be used herein. Chimeric, humanized, veneered, CDR-replaced, reshaped or other recombinant whole or partial antibodies are also suitable.

In the ELISA format, bound MHC molecules can be detected using an antibody or other binding moiety capable of binding MHC molecules. This binding moiety or antibody may be tagged with a detectable label, or may be detected using a detectably labeled secondary antibody or binding reagent. Detectable labels or tags are known in the art, and include fluorescent, calorimetric and radiolabels, for instance.

Other assay strategies can incorporate specific T-cell receptors to screen for their corresponding MHC-peptide complexes, which can be done either in vitro or in vivo. For example, an in vitro anergy assay determines if non-responsiveness has been induced in the T cells being tested. Briefly, an MHC molecule containing antigenic peptide in the peptide binding groove can be mixed with responder cells, preferably peripheral blood mononuclear cells (PBMN) (a heterogeneous population including B and T lymphocytes, monocytes and dendritic cells), PBMNC lymphocytes, freshly isolated T lymphocytes, in vivo primed splenocytes, cultured T cells, or established T cell lines or clones. Responder cells from mammals immunized with, or having a demonstrable cellular immune response to, the antigenic peptide are particularly preferred.

Subsequently, these responder cells are combined with stimulator cells (antigen presenting cells; APCs) that have been pulsed or primed with the same antigenic peptide. In a preferred embodiment, the stimulator cells are antigenic peptide-presenting cells, such as PBMNCs, PBMNCs that have been depleted of lymphocytes, appropriate antigenic peptide-presenting cell lines or clones (such as EBV-transformed B cells), EBV transformed autologous and non-autologous PMNCS, genetically engineered antigen presenting cells, such as mouse L cells or bare lymphocyte cells BLS-1, in particular, DRB1*0401, DRB1*0404 and DRB1*0301 (Kovats et al., J. Exp. Med. 179:2017-22, 1994), or in vivo or in vitro primed or pulsed splenocytes. Stimulator cells from mammals immunized with, or having a demonstrable cellular immune response to, the antigenic peptide are particularly preferred. For certain assay formats, it is preferred to inhibit the proliferation of stimulator cells prior to mixing with responder cells. This inhibition may be achieved by exposure to gamma irradiation or to an anti-mitotic agent, such as mitomycin C, for instance. Appropriate negative controls are also included. (nothing; syngeneic APC; experimental peptide; APC +Peptide; MHC:peptide complex; control peptide +/− APC). Further, to assure that non-responsiveness represents anergy, the proliferation assay may be set up in duplicate, +/− recombinant IL-2 since it has been demonstrated that IL-2, can rescue anergized cells.

After an approximately 72 hour incubation, the activation of responder cells in response to the stimulator cells is measured. In a preferred embodiment, responder cell activation is determined by measuring proliferation using ³H-thymidine uptake (Crowley et al., J. Immunol. Meth. 133:55-66, 1990). Alternatively, responder cell activation can be measured by the production of cytokines, such as IL-2, or by determining the presence of responder cell-specific, and particularly T cell-specific, activation markers. Cytokine production can be assayed by testing the ability of the stimulator +responder cell culture supernatant to stimulate growth of cytokine-dependent cells. Responder cell- or T cell-specific activation markers may be detected using antibodies specific for such markers.

Preferably, the soluble, fused MHC heterodimer:peptide complex induces non-responsiveness (for example, anergy) in the antigenic peptide-reactive responder cells. In addition to soluble, fused MHC heterodimer:peptide complex recognition, responder cell activation requires the involvement of co-receptors on the stimulator cell (the APC) that have been stimulated with co-stimulatory molecules. By blocking or eliminating stimulation of such co-receptors (for instance, by exposing responder cells to purified soluble, fused MHC heterodimer:peptide complex, by blocking with anti-receptor or anti-ligand antibodies, or by “knocking out” the gene(s) encoding such receptors), responder cells can be rendered non-responsive to antigen or to soluble, fused MHC heterodimer:peptide complex.

In a preferred embodiment, responder cells are obtained from a source manifesting an autoimmune disease or syndrome. Alternatively, autoantigen-reactive T cell clones or lines are preferred responder cells. In another preferred embodiment, stimulator cells are obtained from a source manifesting an autoimmune disease or syndrome. Alternatively, APC cell lines or clones that are able to appropriately process and/or present autoantigen to responder cells are preferred stimulator cells. In a particularly preferred embodiment, responder and stimulator cells are obtained from a source with diabetes or multiple sclerosis.

At this point, the responder T cells can be selectively amplified and/or stimulated, thereby producing a subset of T cells that are specific for the antigenic peptide. For instance, antigenic peptide-reactive responder cells may be selected by flow cytometry, and particularly by fluorescence activated cell sorting. This subset of responder cells can be maintained by repetitive stimulation with APCs presenting the same antigenic peptide. Alternatively, responder cell clones or lines can be established from this responder cell subset. Further, this subset of responder cells can be used to map epitopes of the antigenic peptide and the protein from which it is derived.

Other methods to assess the biological activity of the soluble, fused MHC heterodimer:peptide complexes are known in the art and can be used herein, such as using a microphysiometer, to measure production of acidic metabolites in T cells following interaction with antigenic peptide. Other assay methods include competation assays, comparing soluble, fused MHC heterodimer:complex response with that to the normal antigen. Also measurement production of such indicators as cytokines or γ interferon can provide an indication of complex response.

Similar assays and methods can be developed for and used in animal models of diseases mediated by MHC:peptide complexes. For instance, a polynucleotide encoding I-A^{g7} MHC Class II molecules of NOD mice, a model system for insulin-dependent diabetes mellitus (IDDM), can be combined with autoantigenic peptides of GAD to study induction of non-responsiveness in the animal model.

Soluble, fused MHC heterodimer:peptide complex can be tested in vivo in a number of animal models of autoimmune disease. For example, NOD mice are a spontaneous model of IDDM. Treatment with the soluble, fused MHC heterodimer:peptide complex prior to or after onset of disease can be monitored by assay of urine glucose levels in the NOD mouse, as well as by in vitro T cell proliferation assays to assess reactivity to known autoantigens (see Kaufman et al., Nature 366:69-72, 1993, for example). Alternatively, induced models of autoimmune disease, such as EAE, can be treated with relevant soluble, fused heterodimer:peptide complex. Treatment in a preventive or intervention mode can be followed by monitoring the clinical symptoms of EAE.

The NOD mouse strain (H-2g⁷) is a murine model for autoimmune IDDM. In NOD mice, the disease is characterized by anti-islet cell antibodies, severe insulitis, and evidence for autoimmune destruction of beta-cells (see, for instance, Kanazawa et al., Diabetologia 27:113, 1984). The disease can be passively transferred with lymphocytes and prevented by treatment with cyclosporin-A (Ikehara et al., Proc. Natl. Acad. Sci. USA 82:7743-47, 1985; Mori et al., Diabetologia 29:244-47, 1986). Untreated animals develop profound glucose intolerance and ketosis, and succumb within weeks of the onset of the disease. The colony in current use (#11 NOD/CaJ) has a high incidence of diabetes development in males compared to other colonies, 50-65% of males and 90-95% of the females develop diabetes within the first seven months of life (Pozzilli et al., Immunology Today 14:193-96, 1993). Breeding studies have defined at least two genetic loci responsible for disease susceptibility, one of which maps to the MHC. Characterization of NOD class II antigens at both the serological and molecular level suggest that the susceptibility to autoimmune disease is linked to I-A^{g7} (Acha-Orbea and McDevitt, Proc. Natl. Acad. Sci. USA 84:2435-39, 1987).

Development of diabetes can be studied in several ways, for example, by spontaneous disease development or in an adoptive transfer model (Miller et al., J. Immunol. 140:52-58, 1988). NOD mice spontaneously develop autoimmune diabetes. In NOD/CaJ mice, diabetes in females is first observed at 3 months of age. Young NOD/CaJ female mice can be treated with peptide, peptide:MHC complex or a control preparation and then followed for 6 months to see if there is evidence of disease development. NOD mice can be screened for diabetes by monitoring urinary glucose levels, and those animals showing positive urine values are tail clipped and the blood further analyzed for blood glucose with a glucometer. Those mice having blood glucose values of 250 mg/dl or over are classified as overtly diabetic. This method involves treating the autoreactive naive T cell.

IDDM can also be adoptively transferred by transplanting splenic cells from a diabetic-donor to a non-diabetic recipient (Baron et al., J. Clin. Invest. 93:1700-08, 1994). This method involves treating in vivo activated mature T cells. Briefly, NOD/CaJ mice are irradiated (730 rad) and randomly divided into treatment groups. Splenocytes, preferably about 1.5×10⁷, from newly diabetic mice are isolated and injected intravenously into non-diabetic NOD 7-8 week old recipient mice, followed six hours later with intravenous injections of saline, peptide or MHC:peptide complex at 10, 5, or 1 μg/mouse. The injections are repeated on days 4, 8 and 12 following the original injection. Mice are tested for the onset of diabetes by urine analysis, and at the time of sacrifice, blood glucose. Treatment of these mice with an MHC:peptide complex is expected to lengthen the time period before the onset of diabetes and/or to prevent or ameliorate the disease. On the day the first animal shows overt signs of diabetes, mice from each treatment group are randomly selected and sacrificed, and spleens and pancreases are removed for immunohistochemical analysis. The end point of the study is when all of the mice in the control group (saline) develop diabetes. Saline treated mice generally develop diabetes within about 20 days.

Expression systems suitable for production of appropriate soluble, fused MHC heterodimer:peptide complexes are available and known in the art. Various prokaryotic, fungal, and eukaryotic host cells are suitable for expression of soluble, fused MHC heterodimer:peptide complexes.

Prokaryotes that are useful as host cells, according to the present invention, most frequently are represented by various strains of Escherichia coli. However, other microbial strains can also be used, such as bacilli, for example Bacillus subtilis, various species of Pseudomonas, or other bacterial strains.

According to the invention, the soluble, fused MHC heterodimer:peptide complexes are expressed from recombinantly engineered nucleotide sequences that encode the soluble, fused MHC heterodimer:peptide polypeptides by operably linking the engineered nucleic acid coding sequence to signals that direct gene expression in prokaryotes. A nucleic acid is “operably linked” when it is placed into a functional relationship with another nucleic acid sequence. For instance, a promoter or enhancer is operably linked to a coding sequence if it effects the transcription of the sequence. Generally, operably linked means that the nucleic acid sequences being linked are contiguous and, where necessary to join two protein coding regions, contiguous and in reading frame.

The genes encoding the soluble, fused MHC heterodimer:peptide complexes may be inserted into an “expression vector”, “cloning vector”, or “vector”, terms which are used interchangeably herein and usually refer to plasmids or other nucleic acid molecules that are able to replicate in a chosen host cell. Expression vectors may replicate autonomously, or they can replicate by being inserted into the genome of the host cell, by methods well known in the art. Vectors that replicate autonomously will have an origin of replication or autonomous replicating sequence (ARS) that is functional in the chosen host cell(s).

Plasmid vectors that contain replication sites and control sequences derived from a species compatible with the chosen host are used. For example, E. coli is typically transformed using derivatives of pBR322, a plasmid derived from E. coli species by Bolivar et al., Gene 2:95-113, 1977. Often, it is desirable for a vector to be usable in more than one host cell, e.g., in E. coli for cloning and construction, and in a Bacillus cell for expression.

The expression vectors typically contain a transcription unit or expression cassette that contains all the elements required for the expression of the DNA encoding the MHC molecule in the host cells. A typical expression cassette contains a promoter operably linked to the DNA sequence encoding a soluble, fused MHC heterodimer:peptide complex and a ribosome binding site. The promoter is preferably positioned about the same distance from the heterologous transcription start site as it is from the transcription start site in its natural setting. As is known in the art, however, some variation in this distance can be accommodated without loss of promoter function. In addition to a promoter sequence, the expression cassette can also contain a transcription termination region downstream of the structural gene to provide for efficient termination. The termination region may be obtained from the same gene as the promoter sequence or may be obtained from a different gene.

Commonly used prokaryotic control sequences which are defined herein to include promoters for transcription initiation, optionally with an operator, along with ribosome binding site sequences, include such commonly used promoters as the betalactamase (penicillinase) and lactose (lac) promoter systems (Change et al., Nature 198:1056, 1977) and the tryptophan (trp) promoter system (Goeddel et al., Nucleic Acids Res. 8:4057-74, 1980) and the lambda-derived P_{L} promoter and N-gene ribosome binding site (Shimatake et al., Nature 292:128-32, 1981). Any available promoter system that functions in prokaryotes can be used.

Either constitutive or regulated promoters can be used in the present invention. Regulated promoters can be advantageous because the host cells can be grown to high densities before expression of the soluble, fused MHC heterodimer:peptide complexes is induced. High level expression of heterologous proteins slows cell growth in some situations. Regulated promoters especially suitable for use in E. coli include the bacteriophage lambda P_{L} promoter, the hybrid trp-lac promoter (Amann et al., Gene 25:167-78 1983;, and the bacteriophage T7 promoter.

For expression of soluble, fused MHC heterodimer:peptide complexes in prokaryotic cells other than E. coli, a promoter that functions in the particular prokaryotic species is required. Such promoters can be obtained from genes that have been cloned from the species, or heterologous promoters can be used. For example, the hybrid trp-lac promoter functions in Bacillus in addition to E. coli.

A ribosome binding site (RBS) is also necessary for expression of soluble, fused MHC heterodimer:peptide complexes in prokaryotes. An RBS in E. coli, for example, consists of a nucleotide sequence 3-9 nucleotides in length located 3-11 nucleotides upstream of the initiation codon (Shine and Dalgarno, Nature, 254:34-40, 1975; Steitz, In Biological regulation and development: Gene expression (ed. R. F. Goldberger), vol. 1, p. 349, 1979, Plenum Publishing, N.Y.).

Translational coupling may be used to enhance expression. The strategy uses a short upstream open reading frame derived from a highly expressed gene native to the translational system, which is placed downstream of the promoter, and a ribosome binding site followed after a few amino acid codons by a termination codon. Just prior to the termination codon is a second ribosome binding site, and following the termination codon is a start codon for the initiation of translation. The system dissolves secondary structure in the RNA, allowing for the efficient initiation of translation. See Squires, et. al., J. Biol. Chem. 263:16297-16302, 1988.

The soluble, fused MHC heterodimer:peptide complexes can be expressed intracellularly, or can be secreted from the cell. Intracellular expression often results in high yields. However, some of the protein may be in the form of insoluble inclusion bodies. Although some of the intracellularly produced MHC polypeptides of the present invention may active upon being harvested following cell lysis, the amount of soluble, active MHC polypeptide may be increased by performing refolding procedures (see, e.g., Sambrook et al., Molecular Cloning: A Laboratory Manual Second Edition, Cold Spring Harbor, N.Y., 1989.; Marston et al., Bio/Technology 2:800-804, 1985; Schoner et al., Bio/Technology 3:151-54, 1985). Preferrably, for purification and refolding the cell pellet is lysed and refolded in urea-borate-DTT buffer followed by urea-borate buffer and reverse phase HPLC purification using either silica gel based Vydac (Hewlett Packard, Wilmington, Del.) or polymer based Poros-R2 (PerSeptive Biosystems) resins, with bead size varing based on the scale of the culture and is described in further detail below optionally, expecially for large scale refolding, the sample can be ultrafiltered into a urea-borate buffer to which is then added 0.2 μM to 1 mM copper sulfate, preferrably 0.2 to 20 μM, after which folding occurs immediatly. Refolding occures over a range of 0.1 to 2.5 mg/ml protein.

More than one MHC:peptide complex may be expressed in a single prokaryotic cell by placing multiple transcriptional cassettes in a single expression vector, or by utilizing different selectable markers for each of the expression vectors which are employed in the cloning strategy.

A second approach for expressing the MHC:peptide complexes of the invention is to cause the polypeptides to be secreted from the cell, either into the periplasm or into the extracellular medium. The DNA sequence encoding the MHC polypeptide is linked to a cleavable signal peptide sequence. The signal sequence directs translocation of the MHC:peptide complex through the cell membrane. An example of a suitable vector for use in E. coli that contains a promoter-signal sequence unit is pTA1529, which has the E. coli phoA promoter and signal sequence (see, e.g., Sambrook et al., supra; Oka et al., Proc. Natl. Acad. Sci. USA 82:7212-16, 1985; Talmadge et al., Proc. Natl. Acad. Sci. USA 77:39892, 1980; Takahara et al., J. Biol. Chem. 260:2670-74, 1985). Once again, multiple polypeptides can be expressed in a single cell for periplasmic association.

The MHC:peptide complexes of the invention can also be produced as fusion proteins. This approach often results in high yields, because normal prokaryotic control sequences direct transcription and translation. In E. coli, lacZ fusions are often used to express heterologous proteins. Suitable vectors are readily available, such as the pUR, pEX, and pMR100 series (see, e.g., Sambrook et al., supra). For certain applications, it may be desirable to cleave the non-MHC amino acids from the fusion protein after purification. This can be accomplished by any of several methods known in the art, including cleavage by cyanogen bromide, a protease, or by Factor X, (see, e.g. Sambrook et al., supra.; Goeddel et al., Proc. Natl. Acad. Sci. USA 76:106-10, 1979; Nagai et al., Nature 309:810-12, 1984; Sung et al., Proc. Natl. Acad. Sci. USA 83:561-65, 1986). Cleavage sites can be engineered into the gene for the fusion protein at the desired point of cleavage.

Foreign genes, such as soluble, fused MHC heterodimer:peptide complexes, can be expressed in E. coli as fusions with binding partners, such as glutathione-S-transferase (GST), maltose binding protein, or thioredoxin. These binding partners are highly translated and can be used to overcome inefficient initiation of translation of eukaryotic messages in E. coli. Fusion to such binding partner can result in high-level expression, and the binding partner is easily purified and then excised from the protein of interest. Such expression systems are available from numerous sources, such as Invitrogen Inc. (San Diego, Calif.) and Pharmacia LKB Biotechnology Inc. (Piscataway, N.J.).

A method for obtaining recombinant proteins from E. coli which maintains the integrity of their N-termini has been described by Miller et al. Biotechnology 7:698-704 (1989). In this system, the gene of interest is produced as a C-terminal fusion to the first 76 residues of the yeast ubiquitin gene containing a peptidase cleavage site. Cleavage at the junction of the two moieties results in production of a protein having an intact authentic N-terminal reside.

The vectors containing the nucleic acids that code for the soluble, fused MHC heterodimer:peptide complexes are transformed into prokaryotic host cells for expression. “Transformation” refers to the introduction of vectors containing the nucleic acids of interest directly into host cells by well known methods. The particular procedure used to introduce the genetic material into the host cell for expression of the soluble, fused MHC heterodimer:peptide complex is not particularly critical. Any of the well known procedures for introducing foreign nucleotide sequences into host cells may be used. It is only necessary that the particular host cell utilized be capable of expressing the gene.

Transformation methods, which vary depending on the type of the prokaryotic host cell, include electroporation; transfection employing calcium chloride, rubidium chloride calcium phosphate, or other substances; microprojectile bombardment; infection (where the vector is an infectious agent); and other methods. See, generally, Sambrook et al., supra, and Ausubel it al., (eds.) Current Protocols in Molecular Biology, John Wiley and Sons, Inc., NY, 1987. Reference to cells into which the nucleic acids described above have been introduced is meant to also include the progeny of such cells. Transformed prokaryotic cells that contain expression vectors for soluble, fused MHC heterodimer:peptide complexes are also included in the invention.

After standard transfection or transformation methods are used to produce prokaryotic cell lines that express large quantities of the soluble, fused MHC heterodimer:peptide complex polypeptide, the polypeptide is then purified using standard techniques. See, e.g., Colley et al., J. Chem. 64:17619-22, 1989; and Methods in Enzymology, “Guide to Protein Purification”, M. Deutscher, ed., Vol. 182 (1990). The recombinant cells are grown and the soluble, fused MHC heterodimer:peptide complex is expressed. The purification protocol will depend upon whether the soluble, fused MHC heterodimer:peptide complex is expressed intracellularly, into the periplasm, or secreted from the cell. For intracellular expression, the cells are harvested, lysed, and the is recovered from the cell lysate (Sambrook et al., supra) Periplasmic MHC polypeptide is released from the periplasm by standard techniques (Sambrook et al., supra). If the MHC polypeptide is secreted from the cells, the culture medium is harvested for purification of the secreted protein. The medium is typically clarified by centrifugation or filtration to remove cells and cell debris.

The MHC polypeptides can be concentrated by adsorption to any suitable resin (such as, for example, CDP-Sepharose=, Asialoprothrombin-Sepharose 4B, or Q Sepharose, or by use of ammonium sulfate fractionation, polyethylene glycol precipitation, or by ultrafiltration. Other means known in the art may be equally suitable.

Further purification of the MHC polypeptides can be accomplished by standard techniques, for example, affinity chromatography, ion exchange chromatography, sizing chromatography, reverse phase HPLC, or other protein purification techniques used to obtain homogeneity. The purified proteins are then used to produce pharmaceutical compositions.

DNA constructs may also contain DNA segments necessary to direct the secretion of a polypeptide or protein of interest. Such DNA segments may include at least one secretory signal sequence. Secretory signal sequences, also called leader sequences, prepro sequences and/or pre sequences, are amino acid sequences that play a role in secretion of mature polypeptides or proteins from a cell. Such sequences are characterized by a core of hydrophobic amino acids and are typically (but not exclusively) found at the amino termini of newly synthesized proteins. The secretory signal sequence may be that of the protein of interest, or may be derived from another secreted protein (e.g., t-PA, a preferred mammalian secretory leader) or synthesized de novo. The secretory signal sequence is joined to the DNA sequence encoding a protein of the present invention in the correct reading frame. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830). Very often the secretory peptide is cleaved from the mature protein during secretion. Such secretory peptides contain processing sites that allow cleavage of the secretory peptide from the mature protein as it passes through the secretory pathway. An example of such a processing site is a dibasic cleavage site, such as that recognized by the Saccharomyces cerevisiae KEX2 gene or a Lys-Arg processing site. Processing sites may be encoded within the secretory peptide or may be added to the peptide by, for example, in vitro mutagenesis.

Secretory signals include the a factor signal sequence (prepro sequence: Kurjan and Herskowitz, Cell 30: 933-943, 1982; Kurjan et al., U.S. Pat. No. 4,546,082; Brake, EP 116,201), the PHO5 signal sequence (Beck et al., WO 86/00637), the BAR1 secretory signal sequence (MacKay et al., U.S. Pat. No. 4,613,572; MacKay, WO 87/002670), the SUC2 signal sequence (Carlsen et al., Molecular and Cellular Biology 3: 439-447, 1983), the a-1-antitrypsin signal sequence (Kurachi et al., Proc. Acad. Sci. USA 78: 6826-6830, 1981), the a-2 plasmin inhibitor signal sequence (Tone et al., J. Biochem. (Tokyo) 102: 1033-1042, 1987) and the tissue plasminogen activator signal sequence (Pennica et al., Nature +1: 214-221, 1983). Alternately, a secretory signal sequence may be synthesized according to the rules established, for example, by von Heinje (European Journal of Biochemistry 133: 17-21, 1983; Journal of Molecular Biology 184: 99-105, 1985; Nucleic Acids Research 14: 4683-4690, 1986). Another signal sequence is the synthetic signal LaC212 spx (1-47)—ERLE described in WO 90/10075.

Secretory signal sequences may be used singly or may be combined. For example, a first secretory signal sequence may be used in combination with a sequence encoding the third domain of barrier (described in U.S. Pat. No. 5,037,243, which is incorporated by reference herein in its entirety). The third domain of barrier may be positioned in proper reading frame 3′ of the DNA segment of interest or 5′ to the DNA segment and in proper reading frame with both the secretory signal sequence and a DNA segment of interest.

The choice of suitable promoters, terminators and secretory signals for all expression systems, is well within the level of ordinary skill in the art. Methods for expressing cloned genes in Saccharomyces cerevisiae are generally known in the art (see, “Gene Expression Technology,” Methods in Enzymology, Vol. 185, Goeddel (ed.), Academic Press, San Diego, Calif., 1990 and “Guide to Yeast Genetics and Molecular Biology,” Methods in Enzymology, Guthrie and Fink (eds.), Academic Press, San Diego, Calif., 1991; which are incorporated herein by reference). Proteins of the present invention can also be expressed in filamentous fungi, for example, strains of the fungi Aspergillus (McKnight et al., U.S. Pat. No. 4,935,349, which is incorporated herein by reference). Expression of cloned genes in cultured mammalian cells and in E. coli, for example, is discussed in detail in Sambrook et al. (Molecular Cloning: A Laboratory Manual, Second Edition, Cold Spring Harbor, N.Y., 1989; which is incorporated herein by reference). As would be evident to one skilled in the art, one could express the proteins of the instant invention in other host cells such as avian, insect and plant cells using regulatory sequences, vectors and methods well established in the literature.

In yeast, suitable yeast vectors for use in the present invention include YRp7 (Struhl et al., Proc. Natl. Acad. Sci. USA 76: 1035-1039, 1978), YEp13 (Broach et al., Gene 8: 121-133, 1979), POT vectors (Kawasaki et al, U.S. Pat. No. 4,931,373, which is incorporated by reference herein), pJDB249 and pJDB219 (Beggs, Nature 225:104-108, 1978) and derivatives thereof. Preferred promoters for use in yeast include promoters from yeast glycolytic genes (Hitzeman et al., J. Biol. Chem. 255: 12073-12080, 1980; Alber and Kawasaki, J. Mol. Appl. Genet. 1: 419-434, 1982; Kawasaki, U.S. Pat. No. 4,599,311) or alcohol dehydrogenase genes (Young et al., in Genetic Engineering of Microorganisms for Chemicals, Hollaender et al., (eds.), p. 355, Plenum, N.Y., 1982; Ammerer, Meth. Enzymol. 101: 192-201, 1983). Other promoters are the TPI1 promoter (Kawasaki, U.S. Pat. No. 4,599,311, 1986) and the ADH2-4^{c} promoter (Russell et al., Nature 304: 652-654, 1983; Irani and Kilgore, U.S. patent application Ser. No. 07/784,653, CA 1,304,020 and EP 284 044, which are incorporated herein by reference). The expression units may also include a transcriptional terminator such as the TPI1 terminator (Alber and Kawasaki, ibid.).

Yeast cells, particularly cells of the genus Saccharomyces, are a preferred host for use in producing compound of the current invention. Methods for transforming yeast cells with exogenous DNA and producing recombinant proteins therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075, which are incorporated herein by reference. Transformed cells are selected by phenotype determined by a selectable marker, commonly drug resistance or the ability to grow in the absence of a particular nutrient (e.g., leucine). A preferred vector system for use in yeast is the POTI vector system disclosed by Kawasaki et al. (U.S. Pat. No. 4,931,373), which allows transformed cells to be selected by growth in glucose-containing media. A preferred secretory signal sequence for use in yeast is that of the S. cerevisiae MFα1 gene (Brake, ibid.; Kurjan et al., U.S. Pat. No. 4,546,082). Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092, which are incorporated herein by reference) and alcohol dehydrogenase genes. See also U.S. Pat. Nos. 4,990,446; 5,063,154; 5,139,936 and 4,661,454, which are incorporated herein by reference. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986; Cregg, U.S. Pat. No. 4,882,279; and Stroman et al., U.S. Pat. No. 4,879,231.

Other fungal cells are also suitable as host cells. For example, Aspergillus cells may be utilized according to the methods of McKnight et al., U.S. Pat. No. 4,935,349, which is incorporated herein by reference. Methods for transforming Acremonium chrysogenum are disclosed by Sumino et al., U.S. Pat. No. 5,162,228, which is incorporated herein by reference. Methods for transforming Neurospora are disclosed by Lambowitz, U.S. Pat. No. 4,486,533, which is incorporated herein by reference.

Host cells containing DNA constructs of the present invention are then cultured to produce the heterologous proteins. The cells are cultured according to standard methods in a culture medium containing nutrients required for growth of the particular host cells. A variety of suitable media are known in the art and generally include a carbon source, a nitrogen source, essential amino acids, vitamins, minerals and growth factors. The growth medium will generally select for cells containing the DNA construct by, for example, drug selection or deficiency in an essential nutrient which is complemented by a selectable marker on the DNA construct or co-transfected with the DNA construct.

Yeast cells, for example, are preferably cultured in a chemically defined medium, comprising a non-amino acid nitrogen source, inorganic salts, vitamins and essential amino acid supplements. The pH of the medium is preferably maintained at a pH greater than 2 and less than 8, preferably at pH 6.5. Methods for maintaining a stable pH include buffering and constant pH control, preferably through the addition of sodium hydroxide. Preferred buffering agents include succinic acid and Bis-Tris (Sigma Chemical Co., St. Louis, Mo.). Yeast cells having a defect in a gene required for asparagine-linked glycosylation are preferably grown in a medium containing an osmotic stabilizer. A preferred osmotic stabilizer is sorbitol supplemented into the medium at a concentration between 0.1 M and 1.5 M, preferably at 0.5 M or 1.0 M. Cultured mammalian cells are generally cultured in commercially available serum-containing or serum-free media. Selection of a medium appropriate for the particular host cell used is within the level of ordinary skill in the art.

Methods for introducing exogenous DNA into mammalian host cells include calcium phosphate-mediated transfection (wigler et al., Cell 14:725, 1978; Corsaro and Pearson, Somatic Cell Genetics 7:603, 1981; Graham and Van der Eb, Virology 52:456, 1973), electroporation (Neumann et al., EMBO J. 1:841-45, 1982) and DEAE-dextran mediated transfection (Ausubel et al., (eds), Current Protocols in Molecular Biology, John Wiley and Sons, Inc., NY, 1987), which are incorporated herein by reference. Cationic lipid transfection using commercially available reagents, including the Boehringer Mannheim TRANSFECTION-REAGENT (N-[1-(2,3-dioleoyloxy)propyl]-N,N,N-trimethyl ammoniummethylsulfate; Boehringer Mannheim, Indianapolis, Ind.) or LIPOFECTIN reagent (N-[l-(2,3-dioleoyloxy)propyl]-N,N,N-trimethylammonium chloride and dioleoyl phosphatidylethanolamine; GIBCO-BRL, Gaithersburg, Md.) using the manufacturer-supplied directions, may also be used. A preferred mammalian expression plasmid is Zem229R (deposited under the terms of the Budapest Treaty with American Type Culture Collection, 12301 Parklawn Drive, Rockville, Md. on Sep. 28, 1993 as an E. coli HB101 transformant and assigned Accession Number 69447). The production of recombinant proteins in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134, which are incorporated herein by reference. Preferred cultured mammalian cells include the COS-1 (ATCC No. CRL 1650), COS-7 (ATCC No. CRL 1651), BHK (ATCC No. CRL 1632), BHK 570 (ATCC No. CRL 10314), DG44, and 293 (ATCC No. CRL 1573; Graham et al., J. Gen. Virol. 36:59-72, 1977) cell lines. Additional suitable cell lines are known in the art and available from public depositories such as the American Type Culture Collection, Rockville, Md. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978, which are incorporated herein by reference) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems may also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used.

The soluble, fused MHC:peptide complexes of the present invention can be purified by first isolating the polypeptides from the cells followed by conventional purification methods, such as by ion-exchange and partition chromatography as described by, for example, Coy et al. (Peptides Structure and Function, Pierce Chemical Company, Rockford, Ill., pp 369-72, 1983), by reverse-phase chromatography as described, for example, by Andreu and Merrifield (Eur. J. Biochem. 164: 585-90, 1987), or by HPLC as described, for example, by Kofod et al. (Int. J. Peptide and Protein Res. 32: 436-40, 1988). Additional purification can be achieved by additional conventional purification means, such as liquid chromatography, gradient centrifugation, and gel electrophoresis, among others. Methods of protein purification are known in the art (see generally, Scopes, R., Protein Purification, Springer-Verlag, N.Y., 1982, which is incorporated by reference herein) and can be applied to the purification of the recombinant polypeptides described herein. Soluble, fused MHC heterodimer:peptide complexes of at least about 50% purity are preferred, at least about 70-80% purity more preferred, and about 95-99% or more purity most preferred, particularly for pharmaceutical uses. Once purified, either partially or to homogeneity, as desired, the soluble, fused MHC heterodimer:peptide complexes may then be used diagnostically or therapeutically, as further described below.

The soluble, fused MHC heterodimer:peptide complexes of the present invention may be used within methods for down-regulating parts of the immune system that are reactive in autoimmune diseases. The soluble, fused MHC heterodimer:peptide complexes of the present invention are contemplated to be advantageous for use as immunotherapeutics to induce immunological tolerance or nonresponsiveness (anergy) in patients predisposed to mount or already mounting an immune response those particular autoantigens. A patient having or predisposted to a particular autoimmune disease is identified and MHC type is determined by methods known in the art. The patients's T cells can be examined in vitro to determine autoantigenic peptide(s) recognized by the patients's autoreactive T cells using complexes and methods described herein. The patient can then be treated with complexes of the invention. Such methods will generally include administering soluble, fused MHC heterodimer:peptide complex in an amount sufficient to lengthen the time period before onset of the autoimmune disease and/or to ameliorate or prevent that disease. Soluble, fused MHC heterodimer:peptide complexes of the present invention are therefore contemplated to be advantageous for use in both therapeutic and diagnostic applications related to autoimmune diseases.

The therapeutic methods of the present invention may involve oral tolerance (Weiner et al., Nature 376: 177-80, 1995), or intravenous tolerance, for example. Tolerance can be induced in mammals, although conditions for inducing such tolerance will vary according to a variety of factors. To induce immunological tolerance in an adult susceptible to or already suffering from an autoantigen-related disease such as IDDM, the precise amounts and frequency of administration will also vary. For instance for adults about 20-80 μg/kg can be administered by a variety of routes, such as parenterally, orally, by aerosols, intradermal injection, and the like. For neonates, tolerance can be induced by parenteral injection or more conveniently by oral administration in an appropriate formulation. The precise amount administrated, and the mode and frequency of dosages, will vary.

The soluble, fused MHC heterodimer:peptide complexes will typically be more tolerogenic when administered in a soluble form, rather than in an aggregrated or particulate form. Persistence of a soluble, fused MHC heterodimer:peptide complex of the invention is generally needed to maintain tolerance in an adult, and thus may require more frequent administration of the complex, or its administration in a form which extends the half-life of the complex. See-for example, Sun et al., Proc. Natl. Acad. Sci. USA 91: 10795-99, 1994

Within another aspect of the invention, a pharmaceutical composition is provided which comprises a soluble, fused MHC hecerodimer:peptide complex of the present invention contained in a pharmaceutically acceptable carrier or vehicle for parenteral, topical, oral, or local administration, such as by aerosol or transdermally, for prophylactic and/or therapeutic treatment, according to conventional methods. The composition may typically be in a form suited for systemic injection or infusion and may, as such, be formulated with sterile water or an isotonic saline or glucose solution. Formulations may further include one or more diluents, fillers, emulsifiers, preservatives, buffers, excipients, and the like, and may be provided in such forms as liquids, powders, emulsions, suppositories, liposomes, transdermal patches and tablets, for example. One skilled in the art may formulate the compounds of the present invention in an appropriate manner, and in accordance with accepted practices, such as those disclosed in Remington's Pharmaceutical Sciences, Gennaro (ed.), Mack Publishing Co., Easton, Pa. 1990 (which is incorporated herein by reference in its entirety).

Pharmaceutical compositions of the present invention are administered at daily to weekly intervals. An “effective amount” of such a pharmaceutical composition is an amount that provides a clinically significant decrease in a deleterious T cell-mediated-immune response to an autoantigen, for example, those associated with IDDM, or provides other pharmacologically beneficial effects. Such amounts will depend, in part, on the particular condition to be treated, age, weight, and general health of the patient, and other factors evident to those skilled in the art. Preferably the amount of the soluble, fused MHC heterodimer:peptide complex administered will be within the range of 20-80 μg/kg. Compounds having significantly enhanced half-lives may be administered at lower doses or less frequently.

Kits can also be supplied for therapeutic or diagnostic uses. Thus, the subject composition of the present invention may be provided, usually in a lyophilized form, in a container. The soluble, fused MHC heterodimer:peptide complex is included in the kits with instructions for use, and optionally with buffers, stabilizers, biocides, and inert proteins. Generally, these optional materials will be present at less than about 5% by weight, based on the amount of soluble, fused MHC heterodimer:peptide complex, and will usually be present in a total amount of at least about 0.001% by weight, based on the soluble, fused MHC heterodimer:peptide complex concentration. It may be desirable to include an inert extender or excipient to dilute the active ingredients, where the excipient may be present in from about 1 to 99% weight of the total composition.

Within one aspect of the present invention, soluble, fused MHC heterodimer:peptide complexes are utilized to prepare antibodies for diagnostic or therapeutic uses. As used herein, the term “antibodies” includes polyclonal antibodies, monoclonal antibodies, antigen-binding fragments thereof such as F(ab′)₂ and Fab fragments, as well as recombinantly produced binding partners. These binding partners incorporate the variable or CDR regions from a gene which encodes a specifically binding antibody. The affinity of a monoclonal antibody or binding partner may be readily determined by one of ordinary skill in the art (see, Scatchard, Ann. NY Acad. Sci. 51: 660-72, 1949).

Methods for preparing polyclonal and monoclonal antibodies have been well described in the literature (see, for example, Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982, which is incorporated herein by reference). As would be evident to one of ordinary skill in the art, polyclonal antibodies may be generated from a variety of warm-blooded animals, such as horses, cows, goats, sheep, dogs, chickens, rabbits, mice, or rats, for example. The immunogenicity of the soluble, fused MHC heterodimer:peptide complexes may be increased through the use of an adjuvant, such as Freund's complete or incomplete adjuvant. A variety of assays known to those skilled in the art may be utilized to detect antibodies which specifically bind to a soluble, fused MHC heterodimer:peptide complex. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radio-immunoassays, radio-immunoprecipitations, enzyme-linked immuno-sorbent assays, dot blot assays, inhibition or competition assays, and sandwich assays.

Additional techniques for the preparation of monoclonal antibodies may be utilized to construct and express recombinant monoclonal antibodies. Briefly, mRNA is isolated from a B cell population and used to create heavy and light chain immunoglobulin cDNA expression libraries in a suitable vector such as the λIMMUNOZAP(H) and λIMMUNOZAP(L) vectors, which may be obtained from Stratogene Cloning Systems (La Jolla, Calif.). These vectors are then screened individually or are co-expressed to form Fab fragments or antibodies (Huse et-. al., Science 246: 1275-81, 1989; Sastry et al., Proc. Natl. Acad. Sci. USA 86: 5728-32, 1989). Positive plaques are subsequently converted to a non-lytic plasmid which allows high level expression of monoclonal antibody fragments in E. coli.

Antibodies of the present invention may be produced by immunizing an animal selected from a wide variety of warm-blooded animals, such as horses, cows, goats, sheep, dogs, chickens, rabbits, mice, and rats, with a recombinant soluble, fused MHC heterodimer:peptide complex. Serum from such animals are a source of polyclonal antibodies. Alternatively antibody producing cells obtained from the immunized animals are immortalized and screened. As the generation of human monoclonal antibodies to a human antigen, such as a soluble, fused MHC heterodimer:peptide complex, may be difficult with conventional immortalization techniques, it may be desirable to first make non-human antibodies. Using recombinant DNA techniques, the antigen binding regions of the non-human antibodyis transfered to the corresponding site of a human antibody coding region to produce a substantially human antibody molecules. Such methods are generally known in the art and are described in, for example, U.S. Pat. No. 4,816,397, and EP publications 173,494 and 239,400, which are incorporated herein by reference.

In another aspect of the invention, the soluble, fused MHC heterodimer:peptide complexes can be used to clone T cells which have specific receptors for the soluble, fused MHC heterodimer:peptide complex. Once the soluble, fused MHC heterodimer:peptide complex-specific T cells are isolated and cloned using techniques generally available to the skilled artisan, the T cells or membrane preparations thereof can be used to immunize animals to produce antibodies to the soluble, fused MHC heterodimer:peptide complex receptors on T cells. The antibodies can be polyclonal or monoclonal. If polyclonal, the antibodies can be murine, lagomorph, equine, ovine, or from a variety of other mammals. Monoclonal antibodies will typically be murine in origin, produced according to known techniques, or human, as described above, or combinations thereof, as in chimeric or humanized antibodies. The anti-soluble, fused MHC heterodimer:peptide complex receptor antibodies thus obtained can then be administered to patients to reduce or eliminate T cell subpopulations that display such receptor. This T-cell population recognizes and participates in the immunological destruction of cells bearing the autoantigenic peptide in an individual predisposed to or already suffering from a disease, such as an autoimmune disease related to the autoantigenic peptide.

The coupling of antibodies to solid supports and their use in purification of proteins is well known in the literature (see, for example, Methods in Molecular Biology. Vol. 1, Walker (Ed.), Humana Press, N.J., 1984, which is incorporated by reference herein in its entirety). Antibodies of the present invention may be used as a marker reagent to detect the presence of MHC heterodimer:peptide complexes on cells or in solution. Such antibodies are also useful for Western analysis or immunoblotting, particularly of purified cell-secreted material. Polyclonal, affinity purified polyclonal, monoclonal and single chain antibodies are suitable for use in this regard. In addition, proteolytic and recombinant fragments and epitope binding domains can be used herein. Chimeric, humanized, veneered, CDR-replaced, reshaped or other recombinant whole or partial antibodies are also suitable.

The following examples are offered by way of illustration, not by way of limitation.

### EXAMPLES

### Example 1

### Construction of a DNA Sequence Encoding a human Soluble, Fused MHC Heterodimer:Peptide Complex

Plasmid pLJ13 contains the MHC Class II β chain (DR1β*1501) signal sequence; a myelin basic protein encoding sequence (from bp 283 to 345, encoding amino acids DENPVVHFFKNIVTPRTPPPS 82 to 102) (SEQ. ID. NO. 33); a DNA sequence encoding a flexible linker represented by the amino acid sequence (GGGSGGS SEQ. ID. NO. 31); β1 region of Class II MHC DR1β*1501 (SEQ. ID. NO. 50) encoding sequence: a DNA sequence encoding a flexible linker, represented by the amino acid sequence (GASAG SEQ. ID. NO. 29); and an al region of Class II MHC DRA*0101 (SEQ. ID. NO. 51) encoding sequence. This plasmid was designed to direct secretion of a soluble, fused MHC heterodimer, denoted β1α1, to which was attached; at the N terminus of β1, a myelin basic protein peptide that has been implicated in multiple sclerosis (Kamholz et al., Proc. Natl. Acad. Sci. USA 83:4962-66, 1986), thus forming a soluble, fused MHC heterodimer:peptide complex.

To construct pLJ13 (SEQ. ID. NO. 49), PCR was used to introduce a DNA sequence encoding MPB at the junction of the signal sequence and β1β2 sequence of the β chain of DR1β*1501. This was followed by joining the MBP-containing β1 region to the al region through a linker sequence which was introduced by PCR.

As a first step, the cDNA encoding a full length α chain, DRA*0101, and cDNA encoding a full length β chain were inserted into the expression vector pZCEP. DNA encoding these molecules may be isolated using standard cloning methods, such as those described by Maniatis et al. (Molecular Cloning: A Laboratory Manual, Cold Spring Harbor, N.Y., 1982); Sambrook et al., (Molecular Cloning: A Laboratory Manual, Second Edition, Cold Spring Harbor, NY, 1989); or Mullis et al., U.S. Pat. No. 4,683,195, which are incorporated herein by reference.

pZCEP (Jelineck et al., Science, 259: 1615-16, 1993) was digested with Hind III and Eco RI, and a 0.85 kb Hind III-Eco RI fragment comprising the cDNA encoding b chain of DR1β*1501 was inserted. The resulting plasmid was designated pSL1.

pZCEP was digested with Bam HI and XbaI, and a ˜0.7 kb SacI-SSP I fragment, comprising the cDNA encoding a chain of DRA*0101, was isolated by agarose gel electrophoresis, and was inserted along with a polylinker sequence containing Bam HI-SacI and SSP I-XbaI ends (SEQ. ID. NO. ). The resulting plasmid was designated pSL2.

A cloning site in the linker sequence was generated using PCR by amplifying a ˜100 bp Hind III/Cla I fragment containing the signal sequence of Class II b DR1b*1501, to which a sequence encoding the first five amino acids (DPVVH) of MBP (82-104) was joined to the 3′ end of the signal sequence. The DNA sequence encoding the amino acids VH was chosen to create a unique ApaLI site.

A second ClaI/XbaI fragment of ˜750 bp was generated using PCR, which contained a sequence encoding the β1β2 region and transmembrane domain of the Class II β chain DR1β*1501, to which joined a DNA sequence encoding the last two amino acids (GS) of the linker to the 5′ end of the β1 sequence. The DNA sequence encoding the amino acids GS was chosen to create a unique Bam HI site.

The fragments were digested with Hind III/Cla I and Cla I/Xba I, isolated by agarose gel electrophoresis, and inserted into Hind III/Xba I-digested pCZEP. The resulting shuttle plasmid was digested with ApaLI and BamHI, and oligonucleotides encoding the remaining portion of the MBP sequence (represented by the amino acid sequence FFKNIVTPRTPPPS) and the start of the flexible linker GGGSG were inserted. The resulting construct contained the MBP sequence joined to the β1β2 sequence of DR1β*1501 through an intervening linker. The resulting plasmid was designated pSL21.

Alternately, a construct containing the signal sequence of DR1β*1501 attached to the N terminal of the MBP peptide (DENPVVHFFKNIVTPRTPPPS SEQ. ID. NO. 33) which was attached to the N terminal of the DR1⊕*1501 β1 domain via a flexable linker (GGGSGGS SEQ. ID. NO. 31). Six overlapping oligo nucleotides were prepared which would reconstruct the signal sequence, MBP peptide flexable linker and attach to the N terminus of the β1 domain through a unique Bam HI site. The oligos were kinased prior to ligation. For each oligo a 50 ml reaction was prepared containing 50 pmol of the oligo (ZC7639 (SEQ. ID. NO. 2), ZC7665 (SEQ. ID. NO. 6), ZC7663 (SEQ. ID. NO. 4), ZC7640 (SEQ. ID. NO. 3), ZC7666 (SEQ. ID. NO. 7) and ZC7664 (SEQ. ID. NO. 5), 22.4 ml TE, 5 ml TMD, 5 ml ATP and 5 ml kinase. The reaction was incubated for 1 hour at 37° C., followed by a 10 minute incubation at 65° C. The kinased oligos were stored at −20° C. until needed. A 10 ml ligation reaction was then prepared containing 0.5 mg Eco RI-Bam HI lineralized pSL1, 20 pmol each kinased oligonucleotide (ZC7639 (SEQ. ID. NO. 2), ZC7665 (SEQ. ID. NO. 6), ZC7663 (SEQ. ID. NO.4), ZC7640 (SEQ. ID. NO.3), ZC7666 (SEQ. ID. NO. 7) and ZC7664 (SEQ. ID. NO. 5), 1 ml TE, 1 ml TMD, 1 ml ATP and 0.5 ml ligase. The reaction was incubated at 37° C. for 1 hour. One microliter of the ligation was electroporated into DH10B competent cells (GIBCO BRL, Gaithersburg, Md.) according to manufacturer's direction and plated onto LB plates containing 50 mg/ml ampicillin, and incubated overnight. A correct recombinant clone was identified by restriction and sequence analysis and given the designation pSL21.

To create pLJ13, a ˜0.48 kb PCR fragment was generated which encoded the DNA sequence from the signal sequence through the b1 region of pSL21, onto which DNA encoding the sequence of a second flexible linker (represented by the amino acid sequence GASAG (SEQ. ID. NO 29) was joined.

A 100 ml PCR reaction was prepared containing 1 mg full length lineralized DR1β*1501 signal/MBP/linker/β chain (pSL21), 200 pmol ZC7511 (SEQ. ID. NO. 1), 200 pmol ZC8194 (SEQ. ID. NO. 8), 10 ml 10× polymerase buffer, 10 ml dNTPs and 1 wax bead (AmpliWax-, Perkin-Elmer Cetus, Norwalk, Conn.). Following an initial cycle of 95° C. for 5 minutes, 5 U Taq polymerase was added, and the reaction was amplified for 30 cycles of 94° C. for 1 minute, 55° C. for 1 minute, and 72° C. for 1 minute. A DR1β*1501 signal sequence/MBP peptide/linker/β1/linker fragment, comprising the 29 amino acid DR1*1501 β chain signal sequence, the 21 amino acid MBP peptide sequence, a 6 amino acid flexible linker (GGGSGGS SEQ. ID. NO. 31), an 83 amino acid β1 domain, and 5 amino acid flexable linker (GASAG SEQ. ID. NO. 29) was obtained. A band of the predicted size, 374 bp, was isolated by low melt agarose gel electrophoresis.

A second −0.261 kb PCR fragment was created which encoded the al portion of DRA*0101, onto which the DNA encoding the second flexible linker was added to the 5′ end, and a DNA sequence encoding a stop codon added to the 3′ end.

A 100 ml PCR reaction was prepared containing 1 mg full length lineralized DRA*0101 (pSL2), 200 pmol ZC8196 (SEQ. ID. NO. 9), 200 pmol ZC8354 (SEQ. ID. NO.14), 10 ml lox polymerase buffer, 10 ml dNTPs and 1 wax bead (AmpliWax˜, Perkin-Elmer Cetus, Norwalk, Conn.). Following an initial cycle of 95° C. for S minutes, S U Taq polymerase was added, and the reaction was amplified for 30 cycles of 94° C. for 1 minute, 55° C. for 2 minutes, and 72° C. for 3 minutes. A linker/DRA*0101 a1 domain comprising the 5 amino acid flexable linker (GASAG SEQ. ID. NO. 29) attached to the N terminus of the 81 amino acid DRA*0101 al domain on to the C terminal was added a stop codon and a Xba I restriction site was obtained. A band of the predicted size, 261 bp, was isolated by low melt agarose gel electrophoresis.

These two PCR fragments were used to produce a final Hind III/Xba I PCR product which encoded the signal sequence of DR1β*1501 joined to the MPB peptide and linker peptide DNA, followed by β1, which was joined to the 5′ end of α1 through DNA encoding the flexible peptide (GASAG SEQ. ID. NO. 29).

A 100 ml PCR reaction was prepared containing 1 ml signal sequence/MBP/linker/β1/linker fragment, 1 ml linker/al fragment, 200 pmol ZC7511 (SEQ. ID. NO. 1), 200 pmol ZC8196 (SEQ. ID. NO. 9), 10 ml lox polymerase buffer, 10 ml dNTPs and 5 U Taq polymerase. The reaction was carried out for 35 cycles of 94° C. for 1 minute, 50° C. for 1 minute, and 72° C. for 1 minute. The 5 amino acid 3′ linker (GASAG SEQ. ID. NO. 29) of the signal sequence/MBP/linker/β1/linker fragment overlapped with the same 5 amino acid linker of the linker/al fragment joining the β1 and α1 domains in frame via the 5 amino acid linker. The resulting 730 bp MBP-β1α1 PCR product contained a 5′ Hind III site followed by the DR1β*1501 β chain signal sequence, a 21 amino acid MBP peptide DENPVVHFFKNIVTPRTPPPS (SEQ. ID. NO. 33), an 8 amino acid flexible linker (GGGSGGSG) attached to the N terminus of the DR1β*1501 β1 domain which was attached to the N terminus of the DRA*0101, α1 domain by a 5 amino acid linker (GASAG SEQ. ID. NO. 29) and ending with a Xba I restriction site. The MBP β1α1 fragment was introduced into Hind III/XbaI pZCEP. A recombinant clone was identified by restriction and sequence analysis and given the designation pLJ13 (human MBP-β1α1)

### Example 2

### Synthesis of NOD Mouse α and β MHC cDNA

Total RNA was isolated from spleen cells of NOD MOUSE NAME according to the method of Maniatis et al. (Molecular Cloning: A Laboratory Manual, Cold Spring Harbor, N.Y., 1982 and Ausubel et al., eds., Current Protocols in Molecular Biology, John Wiley and Sons, Inc., NY, 1987, incorporated herein by reference, using homogenization in guanidinium thiocynate and CsCl centrifugation. Poly(A)+ RNA was isolated using oligo d(T) cellulose chromatography (Mini-Oligo(dT) Cellulose Spin Column Kit (5 Prime-3 Prime), Boulder, Colo.).

First strand cDNA was synthesized using a Superscript˜ RNase H⁻ Reverse Transcriptase Kit (GIBCO BRL) according to the manufacturer's directions. One microliter of a solution containing 1 mg total NOD RNA was mixed with 1 ml oligo dT solution and 13 ml diethylpyrocarbonate-treated water. The mixture was heated at 70° C. for 10 minutes and cooled by chilling on ice.

First strand cDNA synthesis was initiated by the addition of 4 ml Superscript˜ buffer, 4 ml 0.1 M dithiothreitol, 2 ml deoxynucleotide triphosphate solution containing 10 mM each of DATP, dGTP, dTTP, and dCTP, and 2 ml of 200 U/ml Superscript reverse transcriptase to the RNA-primer mixture. The reaction was incubated at room temperature for 10 minutes, followed by an incubation at 42° C. for 50 minutes, then 70° C. for 15 minutes, then cooled on ice. The reaction was terminated by addition of 1 ml RNase H which was incubated at 37° C. for 20 minutes, then cooled on ice.

Two 100 ml PCR reaction mixtures were then prepared. One reaction amplified the a chain of Class II MHC NOD (IA^{g7}) using primers ZC8198 (SEQ ID NO: 10, antisense α chain primer, Xba I site) and ZC8199 (SEQ ID NO: 11, sense α chain primer, Eco RI site) or the β chain of Class II MHC NOD (IA^{g7}) using primers ZC8206 (SEQ. ID. NO. 12, antisense β chain primer, Xba I site) and ZC8207 (SEQ. ID. NO. 13, sense β chain primer, Eco RI site). In both cases, unique restriction sites, Eco RI at the 5′ end of the fragment and Xba I at the 3′ end, were added to allow cloning into an expression vector. Each reaction mixture contained 10 ml of first strand template, 8 ml 10× synthesis buffer, 100 pmol sense primer, 100 pmol antisense primer, 65 ml dH₂O and 1 wax bead (AmpliWax-, Perkin-Elmer Cetus, Norwalk, Conn.). Following an initial cycle of 95° C. for 5 minutes, 1 U Taq polymerase was added, and the reaction was amplified for 30 cycles of 1 minute at 94° C., 2 minutes at 55° C. and 3 minutes at 72° C. The resulting a chain fragment and b chain fragment were digested with Eco RI-Xba I, treated with RNAse, then isolated by low melt agarose gel electrophoresis and ligated into Eco RI-Xba I linearized pZCEP (Jelineck et al., Science, 259: 1615-16, 1993). The full length β chain pZCEP was designated pLJ12, and the full length a chain pZCEP was designated pLJ11.

### Example 3

### Construction of Mouse Soluble Single Chain MHC Molecules Containing Antigenic Peptide Attached Via a Flexible Linker

I Peptide-β1α1

To create a molecule containing an antigenic peptide attached via a flexible linker to the N terminus of a single chain MHC molecule comprising a bi domain linked to an al domain via a second flexible linker, a four step construction was done.

A. GAD-β1α1 IA^{g7}

1) The β1 domain (SEQ. ID. NO. 43) of the IA^{g7} NOD mouse β chain was isolated from the β2 domain and fused to linker fragments on both the 5′ and 3′ ends using PCR.

A 100 ml PCR reaction was prepared containing 100 ng full length, Eco RI/Xba I lineralized, IA^{g7} b chain, 200 pmol ZC9478 (SEQ. ID. NO. 16), 200 pmol ZC9480 (SEQ. ID. NO. 18), 10 ml 10× polymerase buffer, 10 ml dNTPs and 5 U Taq polymerase. The reaction was carried out for 35 cycles of 94° C. for 1 minute, 50° C. for 1 minute, and 72° C. for 1 minute. A β1/linker fragment, comprising the 91 amino acid b1 domain, and 8 amino acid portion of a flexible linker (GGSGGGGS SEQ. ID. NO. 34), fused to the 5′ end, and a 5 amino acid flexible linker (GGSGG SEQ. ID. NO. 30), fused to the 3′ end was obtained. A band of the predicted size, 330 bp, was isolated by low melt agarose gel electrophoresis.

2) A GAD 65 peptide (SRLSKVAPVIKARMMEYGTT (SEQ. ID. NO. 59) and an additional linker fragment were added to the b1/linker fragment from 1 using PCR. In addition, a unique Bam HI site and a the last 16 nucleotides of the phi 10 coupler, adding a second ribosome binding site followed by a stop codon (RBS SEQ. ID. NO. 48) were also added to the 5′ end of the GAD peptide to facilitate cloning and expression.

A 100 ml PCR reaction was prepared using 1 ml of eluted b1/linker fragment from above, 200 pmol ZC9473 (SEQ. ID. NO. 15), 200 pmol ZC9479 (SEQ. ID. NO.17), 200 pmol ZC9480 (SEQ. ID. NO. 18), 10 ml 10× polymerase buffer, 10 ml dNTPs, and 5 U Taq polymerase. The reaction was carried out for 35 cycles of 94° C. for 1 minute, 50° C. for 1 minute, and 72° C. for 1 minute. The fragments were designed so that all contained overlapping 5′ and/or 3′ segments, and could both anneal to their complement strand and serve as primers for the reaction. The final 15 3′ nucleotides of ZC9499 (SEQ. ID. NO. 23) overlap with the first 15 nucleotides of the β1/linker fragment (ggaggctcaggagga) (SEQ. ID. NO. 35), seamlessly joining the GAD peptide in frame with the β1 domain through a 15 amino acid flexible linker (GGGGSGGGGSGGGGS) (SEQ ID. NO.36). ZC9479 (SEQ. ID. NO. 17) served as the 5′ primer, adding a Bam HI site followed by a RBS (SEQ. ID. NO. 48) to the 5′ end of the GAD peptide sequence. A 15 nucleotide overlap (gaggatgattaaatg) between the 3′ end of ZC9479 (SEQ. ID. NO. 17) and the first 15 nucleotides of ZC9473 (SEQ. ID. NO. 15) added the sites in frame with the peptide. The resulting 450 bp GAD/β1 fragment was isolated by low melt agarose gel electrophoresis.

3) The al domain (SEQ. ID. NO. 44) of the IA^{g7} was isolated from the α2 domain, and fused to a linker fragment on the 5′ end and a serine residue, followed by a Spe I and Eco RI site, on the 3′ end using PCR.

A 100 ml PCR reaction was prepared containing 100 ng full length, Eco RI/Xba I lineralized, I-A^{g7} a chain, 200 pmol ZC9481 (SEQ. ID. NO. 19), 200 pmol ZC9493 (SEQ. ID. NO.20), 10 ml 10× polymerase buffer, 10 ml dNTPs, and 5 U Taq polymerase. The reaction was carried out for 35 cycles of 94° C. for 1 minute, 53° C. for 1 minute, and 72° C. for 1 minute. An a1/linker fragment, comprising the 87 amino acid al domain with a 5 amino acid flexible linker (GGSGG) (SEQ. IN. NO. 30), fused to the 5′ end and a serine residue, Spe I and Eco RI site, fused to the 3′ end, was obtained. A band of the predicted size, 300 bp, was isolated by low melt agarose gel electrophoresis.

4) To complete the construct, a final 100 ml PCR reaction was prepared containing 2 ml GAD/β1 fragment from 2), 2 ml α1/linker fragment from 3), 200 pmol ZC9479 (SEQ. ID. NO. 17), 200 pmol ZC9493 (SEQ. ID. NO. 20), 10 ml 10× polymerase buffer, 10 ml dNTPs and 5 U Taq polymerase. The reaction was carried out for 35 cycles of 94° C. for 1 minute, 53° C. for 1 minute, and 72° C. for 1 minute. The 5 amino acid 3′ linker (GGSGG SEQ. ID. NO. 30) of the GAD/β1 fragment overlapped with the 5 amino acid linker of the α1/linker fragment joining the β1 and α1 domains in frame via the 5 amino acid linker. The resulting GAD-β1α1 PCR product contained a 5′ Bam HI site followed by a RBS (SEQ. ID. NO. 48), a 20 amino acid GAD65 peptide (SRLSKVAPVIKARMMEYGTT (SEQ. ID. NO. ), a 15 amino acid flexible linker (GGGGSGGGGSGGGGS (SEQ. ID. NO. 36) attached to the N terminus of the β1 domain of IA^{g7} which was attached to the N terminus of the al domain of IA^{g7} by a 5 amino acid linker (GGSGG SEQ. IS. NO. 30) and ending with a Spe I and Eco RI restriction site. The GAD-β1α1 fragment was restriction digested with Bam HI and Eco RI and isolated by low melt agarose gel electrophoresis. The restriction digested fragments were then subcloned into a Bam HI-Eco RI lineralized expression vector p27313 (WO 95/11702). A correct recombinant clone was identified by restriction and sequence analysis and given the designation pLJ18 (GAD-β1α1 IA^{g7} SEQ. ID. NO. 42).

B) MBP-β1α1 IA^{S}

The β1 domain (SEQ. ID. NO. 46) of IA^{s} was isolated from the β2 domain and fused to linker fragments on both the 5′ and 3′ ends using PCR.

1) A 100 ml PCR reaction was prepared containing 100 ng full length, Eco RI/Xba I lineralized, IAS P chain (p40553), 200 pmol ZC9478 (SEQ. ID. NO. 16), 200 pmol ZC9497 (SEQ. ID. NO. 22), 10 ml 10× polymerase buffer, 10 ml dNTPs and 5 U Taq polymerase. The reaction was carried out for 35 cycles of 94° C. for 1 minute, 53° C. for 1 minute, and 72° C. for 1 minute. An IA^{s}β1/linker fragment, comprising the 91 amino acid β1 domain, with 8 amino acids of a flexable linker (GGSGGGGS SEQ. ID. NO. 34), fused to the 5′ end, and a 5 amino acid flexable linker (GGSGG SEQ. ID. NO. 30), fused to the 3′ end, was obtained. A band of the predicted size, 330 bp, was isolated by low melt agarose gel electrophoresis.

2) A mylein basic protein (MBP) peptide (FFKNIVTPRTPPP SEQ. ID. NO. 37), and the remainder of the 5′ linker, were added using PCR to the IA^{s} β1/linker fragment from above. In addition, a unique Bam HI site, and a ribosome binding site with stop codon (RBS SEQ. ID. NO. 48) were also added to the 5′ end of the MBP peptide to facilitate cloning and expression.

A 100 ml PCR reaction was set up using 1 ml of eluted IA^{s} β1/linker fragment from 1), 200 pmol ZC9499 (SEQ. ID. NO. 23), 200 pmol ZC9479 (SEQ. ID. NO. 17), 200 pmol ZC9497 (SEQ. ID. NO.22), 10 ml 10× polymerase buffer, 10 ml dNTPs, 5 U Taq polymerase. The reaction was carried out for 35 cycles of 94° C. for 1 minute, 50° C. for 1 minute, and 72° C. for 1 minute. The fragments were designed so that all contained overlapping 5t and/or 3′ segments and could both anneal to their complement strand, and serve as primers for the reaction. The final 15 3′ nucleotides of ZC9499 (SEQ. ID. NO. 23) (ggaggctcaggagga SEQ. ID. NO. 35) overlap with the first 15 nucleotides of the IA^{s} b1/linker fragment seamlessly, joining the MBP peptide to the IA^{s} β1 domain through a 15 amino acid flexible linker (GGGGSGGGGSGGGGS SEQ. ID. NO. 36). ZC9479 (SEQ. ID. NO. 17) served as the 5′ primer, completely overlapping the first 32 nucleotides of ZC9499 (SEQ. ID. NO. 23), creating a Bam HI restriction site, and adding a RBS (SEQ. ID. NO. 48) and stop codon in frame with the MBP peptide. The resulting 400 bp MBP/IA^{s} β1 fragment was isolated by low melt agarose gel electrophoresis.

3) The α1 domain (SEQ. ID. NO. 47) of IA^{s} was isolated from the α2 domain and fused to a linker fragment on the 5′ end, and a serine residue, followed by a Spe I and Eco RI site on the 3′ end, using PCR.

A 100 ml PCR reaction was prepared containing 100 ng full length lineralized I-A^{s} α chain (p28520), 200 pmol ZC9481 (SEQ. ID. NO. 19), 200 pmol ZC9496 (SEQ. ID. NO. 21), 10 ml 10× polymerase buffer, 10 ml dNTPs and 5 U Taq polymerase. The reaction was carried out for 35 cycles of 94° C. for 1 minute, 53° C. for 1 minute, and 72° C. for 1 minute. An IAS al/linker fragment, comprising the 87 amino acid IA^{s} α1 domain, with a 5 amino acid flexable linker (GGSGG SEQ. ID. NO. 30), fused to the 5′ end, and a serine residue, Spe I and Eco RI site, fused to the 31 end, was obtained. A band of the predicted size, 300 bp, was isolated by low melt agarose gel electrophoresis.

4) To complete the construct, a final 100 ml PCR reaction was prepared containing 2 ml MBP/IA^{s} β1 fragment from 2), 2 ml IA^{s} α1/linker fragment from 3), 200 pmol ZC9479 (SEQ. ID. NO. 17), 200 pmol ZC9496 (SEQ. ID. NO.21 ), 10 ml 10× polymerase buffer, 10 ml dNTPs and 5 U Taq polymerase. The reaction was carried out for 35 cycles of 94° C. for 1 minute, 53° C. for 1 minute, and 72° C. for 1 minute. The 5 amino acid 3′ linker (GGSGG SEQ. ID. NO. 30) of the MBP/IA^{s} ⊕1 fragment, overlapped with the same 5 amino acid linker of the IA^{s} α1/linker fragment, joining the IA^{s} β1 and IA^{s} α1 domains in frame, via the 5 amino acid linker. The resulting 673 bp MBP-β1α1 IA^{s} PCR product contained a 5′ Bam HI site, followed by a RBS (SEQ. ID. NO. 48), a 13 amino acid MBP peptide (FFKNIVTPRTPPP SEQ. ID. NO. 37), a 15 amino acid flexable linker (GGGGSGGGGSGGGGS SEQ. ID. NO. 36) attached to the N terminus of the IA^{s} 1 domain, which was attached to the N terminus of the IA^{s} α1 domain by a 5 amino acid linker (GGSGG SEQ ID NO 30), and ending with a Spe I and Eco RI restriction site. The MBP β1α1 fragment was restriction digested with Bam HI and Eco RI, and isolated by low melt agarose gel electrophoresis. The restriction digested fragments were then subcloned into a Bam HI-Eco RI lineralized expression vector p27313 (WO 95/11702). A recombinant clone was identified by restriction and sequence analysis and given the designation pLJ19 (MBP β1α1IA^{s} SEQ. ID. NO. 45).

II. Peptide-β1α1α2β2

To create a molecule containing an antigenic peptide, attached via a flexible linker to the N terminus of a single chain MHC molecule, comprising a pi domain, linked to the N terminus of an α1α2 domain, via a flexible linker, which is attached to the N terminus of a β2 domain by a second flexible linker, a four step construction was done.

A. GAD-β1α1α2β2IA^{g7}

1) The α1α2 domain of the I-A^{g7} was fused to a 5 amino acid linker on the 5′ end, and a 15 amino acid linker on the 3′ end, using PCR.

A 100 ml PCR reaction was prepared containing 100 ng full length linearlized I-A^{g7} α chain (pLJ11), 200 pmol ZC9481 (SEQ. ID. NO. 19), 200 pmol ZC9722 (SEQ. ID. NO. 27), 5 ml 10× polymerase buffer, 5 ml dNTPs and 2.5 U Taq polymerase. The reaction was carried out for 35 cycles of 94° C. for 1 minute, 54° C. for 1 minute, and 72° C. for 2 minutes. An I-A^{g7} linker/α1α2/linker fragment, comprising the I-A^{g7} α1α2 domain with a 5 amino acid flexible linker (GGSGG SEQ. ID. NO. 30), fused to the 5′ end, and a 15 amino acid flexable linker (GGGGSGGGGSGGGGS SEQ. ID. NO. 36), fused to the 3′ end, was obtained. A band of the predicted size was isolated by low melt agarose gel electrophoresis.

2) The β2 domain of the I-A^{g7} was isolated from the β1 domain and a 15 amino acid linker was fused to the 5′ end of the β2 domain, and a stop codon followed by an Eco RI restriction site on the 3′ end, using PCR.

A 100 ml PCR reaction was prepared containing 100 ng full length lineralized I-A^{g7} β chain (pLJ12), 200 pmol ZC9721 (SEQ. ID. NO. 26), 200 pmol ZC9521 (SEQ. ID. NO. 24), 5 ml 10× polymerase buffer, 5 ml dNTPs and 2.5 U Taq polymerase. The reaction was carried out for 35 cycles of 94° C. for 1 minute, 54° C. for 1 minute, and 72° C. for 2 minutes. An I-A^{g7} linker/β2 fragment, comprising the β2 domain (SEQ. ID. NO. 58), with a 15 amino acid flexible linker (GGGGSGGGGSGGGGS SEQ. ID. NO.36) fused to the 5′ end, and stop codon and Eco RI restriction site fused to the 3′ end, was obtained. A band of the predicted size was isolated by low melt agarose gel electrophoresis.

3) The α1α2 domain (SEQ. ID. NO. 57)of the I-A^{g7} was fused to β2 domain of I-A^{g7} using PCR. The 15 amino acid linker sequence on the 3′ end of the α1α2 fragment overlapped completely with the same 15 amino acid sequence on the 5′ end of the β2 fragment, joining the domains in frame, via a flexible linker.

A 100 ml PCR reaction was prepared containing 5 ml I-A^{g7} linker/α1α2/linker fragment from 2), 5 ml I-A^{g7} linker/β2 fragment from 3), 200 pmol ZC9481 (SEQ. ID. NO. 19), 200 pmol ZC9721 (SEQ. ID. NO. 26), 10 ml 10× polymerase buffer, 10 ml dNTPs and 5 U Taq polymerase. The reaction was carried out for 30 cycles of 94° C. for 1 minute, 60° C. for 1 minute, and 72° C. for 2 minutes. An I-A^{g7} linker/α1α2/linker/b2 fragment was obtained, comprising the I-A^{g7} α1α2 domain, with a 5 amino acid flexible linker (GGSGG SEQ. ID. NO. 30) fused to the 5′ end, and a 15 amino acid flexible linker (GGGGSGGGGSGGGGS SEQ. ID. NO. 36), fused to the 3′ end, joining it with the 5′ end of the β2 domain. A band of the predicted size was isolated by low melt agarose gel electrophoresis.

4) To complete the construct a final 100 ml PCR reaction was prepared containing 5 ml GAD-β1α1 fragment from A-4 above, 5 ml I-A^{g7} linker/α1α2/linker/β2 fragment from 3), 200 pmol ZC9521 (SEQ. ID. NO. 24), 200 pmol ZC9479 (SEQ. ID. NO. 17), 10 ml 10× polymerase buffer, 10 ml dNTPs and 5 U Taq polymerase. The reaction was carried out for 30 cycles of 94° C. for 1 minute, 60° C. for 1 minute, and 72° C. for 2 minutes. The entire linker/a1 portions of both the GAD-β1α1 and linker/α1α2/linker/β2 fragments overlapped, joining the I-A^{g7} β1 and I-A^{g7} α1α2/linker/β2 domains in frame, via the 5 amino acid flexible linker (GGSGG SEQ. ID. NO. 30). The resulting GAD-β1α1α2β2 I-A^{g7} PCR product contained a 5′ Bam HI site, followed by a RES (SEQ. ID. NO. 48), a 20 amino acid GAD peptide (SRLSKVAPVIKARMMEYGTT (SEQ. ID. NO. 59), a 15 amino acid flexible linker (GGGGSGGGGSGGGGS SEQ. ID. NO.36), attached to the N terminus of the I-A^{g7} β1 domain, which was attached to the N terminus of the α1α2 domain by a 5 amino acid flexible linker (GGSGG, SEQ. ID. NO. 30), and ending with the β2 domain, and an Eco RI restriction site. The GAD-β1α1α2β2 fragment was restriction digested with Bam HI and Eco RI and isolated by low melt agarose gel electrophoresis. The restriction digested fragment was then subcloned into a Bam HI-Eco RI lineralized expression vector p27313 (WO 95/11702). A recombinant clone was identified by restriction and sequence analysis and given the designation pLJ23 (GAD-β1α1α2β2 I-A^{g7} SEQ. ID. NO. 56).

B. MBP-β1α1α2β2 IA^{s}

1) The α1α2 domain of the IA^{s} was fused to a 5 amino acid linker on the 5′ end, and a 15 amino acid linker on the 3′ end, using PCR.

A 100 ml PCR reaction was prepared containing 100 ng full length linearlized I-A^{s} a chain (p28520), 200 pmol ZC9481 (SEQ. ID. NO. 19), 200 pmol ZC9722 (SEQ. ID. NO. 27), 10 ml 10× polymerase buffer, 10 ml dNTPs and 5 U Taq polymerase. The reaction was carried out for 35 cycles of 94° C. for 1 minute, 54° C. for 1 minute, and 72° C. for 2 minutes. An IA^{s} linker/α1α2/linker fragment, comprising the 196 amino acid IA^{s} α1α2 domain, with a 5 amino acid flexible linker (GGSGG SEQ. ID. NO. 30) fused to the 5′ end, and a 15 amino acid flexible linker (GGGGSGGGGSGGGGS SEQ. ID. NO. 36), fused to the 3′ end, was obtained. A band of the predicted size, 650 bp, was isolated by low melt agarose gel electrophoresis.

2) The β2 domain of the IA^{s} was isolated from the b1 domain and fused to a 15 amino acid linker was fused to the 5′ end and a stop codon followed by an Eco RI restriction site on the 3′ end, using PCR.

A 100 ml PCR reaction was prepared containing 100 ng full length lineralized IA^{s} β chain (p40553), 200 pmol ZC9721 (SEQ. ID. NO. 26), 200 pmol ZC9521 (SEQ. ID. NO. 24), 10 ml 10× polymerase buffer, 10 ml dNTPs and 5 U Taq polymerase. The reaction was carried out for 35 cycles of 94° C. for 1 minute, 54° C. for 1 minute, and 72° C. for 2 minutes. An IA^{s} linker/β2 fragment, comprising the 105 amino acid β2 domain (SEQ. ID. NO. 55), with a 15 amino acid flexible linker (GGGGSGGGGSGGGGS SEQ. ID. NO.36) fused to the 5′ end, and stop codon, and Eco RI restriction site, fused to the 3′ end, was obtained. A band of the predicted size, 374 bp, was isolated by low melt agarose gel electrophoresis.

3) The α1α2 domain of the IA^{s} was fused to β2 domain of IA^{s} using PCR. The 15 amino acid linker sequence on the 3 end of the α1α2 fragment overlapped completely with the same 15 amino acid sequence on the 5′ end of the β2 fragment, joining the domains in frame via a flexible linker.

A 100 ml PCR reaction was prepared containing 5 ml IA^{s} linker/α1α2/linker fragment from 2), 5 ml IAS linker/β2 fragment from 3), 200 pmol ZC9481 (SEQ. ID. NO. 19), 200 pmol ZC9721 (SEQ. ID. NO. 26), 10 ml 10× polymerase buffer, 10 ml dNTPs and 5 U Taq-polymerase. The reaction was carried out for 30 cycles of 94° C. for 1 minute, 54° C. for 1 minute, and 72° C. for 2 minutes. An IAS linker/α1α2/linker/β2 fragment was obtained, comprising the 196 amino acid IA^{s} α1α2 domain, with a 5 amino acid flexible linker (GGSGG SEQ. ID. NO. 30) fused to the 5′ end, and a 15 amino acid flexible linker (GGGGSGGGGSGGGGS SEQ. ID. NO. 36), fused to the 3′ end, joining it with the 5′ end of the 106 amino acid β2 domain. A band of the predicted size, 977 bp, was isolated by low melt agarose gel electrophoresis.

4) To complete the construct a final 100 ml PCR reaction was prepared containing 2 ml MBP-β1α1 fragment from B-4 above, 2 ml IA^{s} linker/α1α2/linker/β2 fragment from 3), 200 pmol ZC9521 (SEQ. ID. NO. 24), 200 pmol ZC9479 (SEQ. ID. NO. 17), 10 ml 10× polymerase buffer, 10 ml dNTPs and 5 U Taq polymerase. The reaction was carried out for 30 cycles of 94° C. for 1 minute, 54° C. for 1 minute, and 72 OC for 2 minutes. The entire linker/al portions of both the MBP-β1α1 and linker/α1α2/linker/β2 fragments overlapped, joining the IA^{s} β1 and IA^{s} α1α2/linker/β2 domains, in frame via the 5 amino acid flexible linker (GGSGG SEQ. ID. NO. 30). The resulting 1360 bp MBP-β1α1α2β2 IA^{s} PCR product contained, a 5′ Bam HI site, followed by a RBS (SEQ. ID. NO.48), a 13 amino acid MBP peptide (FFKNIVTPRTPPP SEQ. ID. NO.37), a 15 amino acid flexible linker (GGGGSGGGGSGGGGS SEQ. ID. NO. 36), attached to the N terminus of the IA^{s} β1 domain, which was attached to the N terminus of the full length IA^{s} α domain by a 5 amino acid flexable linker (GGSGG SEQ. ID. NO. 30), and ending with the β2 domain and an Eco RI restriction site. The MBP β1α1α2β2 fragment was restriction digested with Bam HI and Eco RI and isolated by low melt agarose gel electrophoresis. The restriction digested fragment was then subcloned into a Bam HI-Eco RI lineralized expression vector p27313 (WO 95/11702). A recombinant clone was identified by restriction and sequence analysis and given the designation pLJ20 (MBP β1α1α2β2IA^{s} SEQ. ID. NO. 54).

III MBP-α1α2

To create a molecule containing an antigenic peptide attached via a flexable linker to the N terminus of a single chain MHC molecule comprising an α1α2 domain a two step process was done.

1) The α1α2 domain of the I-A^{s} (SEQ. ID. NO. 53) was fused to a 25 amino acid linker on the 5′ end, and a stop codon and Spe I and Eco RI restriction sites on the 3′, end using PCR.

A 100 ml PCR reaction was prepared containing 100 ng full length Eco RI-Xba I lineralized I-A^{s} α chain (p28520), 200 pmol ZC9720 (SEQ. ID. NO. 25), 200 pmol ZC9723 (SEQ. ID. NO. 28), 10 ml 10× polymerase buffer, 10 ml dNTPs and 5 U Taq polymerase. The reaction was carried out for 35 cycles of 94° C. for 1 minute, 54° C. for 1 minute, and 72° C. for 2 minutes. An IA^{s} linker/α1α2 fragment, comprising the 196 amino acid IA^{s} α1α2 domain with a 25 amino acid flexable linker (GGGGSGGGGSGGGGSGGGGSGGGGS SEQ. ID. NO. 32) fused to the 5′ end, and a stop codon and Spe I and Eco RI restriction sites fused to the 3′ end, was obtained. A band of the predicted size, 672 bp, was isolated by low melt agarose gel electrophoresis.

2) A 100 ml PCR reaction was prepared containing 5 ml linker/α1α2 I-A^{s} from 1), 200 pmol ZC9723 (SEQ. ID. NO. 28), 400 pmol ZC9499 (SEQ. ID. NO. 23), 200 pmol ZC9479 (SEQ. ID. NO. 17), 10 ml 10× polymerase buffer, 10 ml dNTPs and S U Taq polymerase. The reaction was carried out for 30 cycles of 94° C. for 1 minute, 54° C. for 1 minute, and 72° C. for 2 minutes. An IA^{s} MBP/linker/α1α2 fragment, comprising the 196 amino acid IA^{s} α1α2 domain with a 25 amino acid flexable linker (GGGGSGGGGSGGGGSGGGGSGGGGS SEQ. ID. NO. 32) fused to the 5′ end, and a stop codon and Spe I and Eco RI restriction sites fused to the 3′ end, was obtained.

There was a 12 amino acid overlap (GGGGSGGGGSGG SEQ. ID. NO. 38) between the 5′ end of the 25 amino acid linker, of the linker/α1α2 fragment, and the 3′ end of ZC9499 (SEQ. ID. NO.23). ZC9499 (SEQ. ID. NO.23) added a Bam HI restriction site, RBS (SEQ. ID. NO. 48), and MBP peptide(FFKNIVTPRTPPP (SEQ. ID. NO. 37), to the 5′ end of the 25 amino acid flexable linker. ZC9479 (SEQ. ID. NO. 17) served as a 5′ primer, overlapping the first 32 nucleotides of ZC9499 (SEQ. ID. NO.23). The resulting 743 bp MBP-α1α2 IA^{s} PCR product contained, a 5′ Bam HI site, followed by a RBS (SEQ. ID. NO. 48), a 13 amino acid MBP peptide (FFKNIVTPRTPPP (SEQ. ID. NO. 37), a 25 amino acid flexable linker (GGGGSGGGGSGGGGSGGGGSGGGGS SEQ. ID. NO. 32) attached to the N terminus of the IA^{s} α1α2 domain, which ended with a Spe I and Eco RI restriction site. The MBP-α1α2 fragment was restriction digested with Bam HI and Eco RI, and isolated by low melt agarose gel electrophoresis. The restriction digested fragment was then subcloned into a Bam HI-Eco RI lineralized expression vector p27313 (WO 95/11702). A recombinant clone was identified by restriction and sequence analysis and given the designation pLJ21 (MBP-α1α2 IA^{s} SEQ. ID. NO. 52).

### Example 4

### Transfection and Induction of Soluble. Fused MHC Heterodimer:Peptide Complexes in E. coli

Transfection

E. coli K-12 strain W3110, was obtained from the ATCC, and was made lysogenic for the phage lambda-DE3 (which carries a copy of the T7 RNA polymerase gene) using the DE3 lysogenization kit from Novagen (Madison, Wis.), following the manufacturer's instructions. Plasmids pLJ18 (GAD β1α1 IA^{g7}), pLJ23 (GAD β1α1α2β2 IA^{g7}), pLJ19 (MBP β1α1 IA^{s}), pLJ20 and (MBP β1α1α2β2 IA^{s}) were transformed into the host strain W3110/DE3 using Ca++ transformation according Maniatis et al. (Molecular Cloning: A Laboratory Manual, Cold Spring Harbor, N.Y., 1982.

Induction

All four plasmid trasfectants were induced as described below. pLJ18 will be used as a prototypical example. Single colonies containing pLJ18 (GAD β1α1 IA^{g7}) were used to inoculate 5-6 ml LB containing 50 mg/ml carbenicillin (Sigma), and the cultures were rotated at 37° C. until the OD₆₀₀ of the culture was between 0.45 and 0.60, usually 3 hours. A glycerol stock was made from a portion of each culture, and 1 ml of culture was spun at 5,000 ×g for 5 minutes at 4° C. To initiate induction, isopropyl-b-b-D-thio-galactopyranoside (IPTG) was added to a final concentration of 1 mM and the cultures were rotated at 37° C. An aliquot was taken from each culture at timepoints 0, 1, 2, and 3 hours, and overnight and the OD₆₀₀ determined. The aliquots were harvested by centrifugation at 5000 × g at 4° C. for 5 minutes. The pellets were resuspended in TE (10 mM Tris-HCl, 1 mM EDTA, pH 8.0) in a volume appropriate to yield 0.02 OD₆₀₀/ml. The timepoint aliquots were then stored at −20° C. until needed.

Fifty microliters from each time point aliquot were electrophoresed on a 4-20% Tris-glycine SDS polyacrylamide gel in denaturing (reducing) sample buffer, followed by Coomassie Blue staining. A band was present at about 33 kD.

For Western blot analysis, a 1/60 dilution of each timepoint aliquot was electrophoresed on a 4-20% Tris-glycine SDS polyacrylamide gel in denaturing (reducing) sample buffer. Proteins were transferred to nitrocellulose by electroblotting. Proteins were visualized by reacting the blots with mouse anti-IA^{g7} MHC antisera, followed by rabbit anti-mouse antibody/horseradish peroxidase conjugate (BioSource International, Camarillo, Calif.) and ECL™ detection reagents (Amersham Corp.). The blots were then exposed to autoradiography film. A band was present at about 33 kD.

### Example 5

### Purification From Inclusion Bodies and Refolding of GAD-β1-α1

A 2 liter culture of GAD-β1-α1 was grown at 37° C. with shaking until an OD₆₀₀ of 0.77 were obtained. Initial culture volumes can be scaled up for large scale production of the protein. Induction was initiated by the addition of IPTG to a final concentration of 1 mM. The cultures were grown for 3 hours 15 minutes following induction, until an OD₆₀₀ of 0.97 was achieved. Whole cell pellets were stored in 20 ml TE (50 mM Tris-HCl, pH 8.0, 2 mM EDTA) at −20° C. until needed.

The pellet was resuspended in 1/10 initial culture volume of TE, 100 mg/ml lysozyme and 0.1% Triton X-100 and incubated at 30° C. for 20 minutes, followed by a cool down on ice, then sonicated with three 20 second pulses on power setting 5 (Branson 450) with gentle mixing between pulses.

The pellet lysate was then spun in an SS34 rotor at 12,000 × g for 10 minutes at 4° C. The pellet was washed in 1/10 initial culture volume of 1% NP-40 in TEN (50 mM Tris-HCl pH 8.0, 2 mM EDTA, 100 mM NaCl) and spun in SS34 rotor at 12,000 × g for 10 minutes at 4° C. The pellet was then washed in 1/10 initial culture volume TEN containing no detergent. The pellet was spun as before, the supernatant discarded. The pellet was resuspended in extraction buffer (8 M urea, 25 mM borate pH 8.5, 10 mM DDT) to a concentration of approximately 200 mg/ml and incubated at 37° C. for about 2 hours. An additional 38 ml urea/borate/DTT buffer was added to the supernatant and the entire sample was dialyzed against 3.5 L 4 M urea, 50 mM borate pH 8.1 at 4° C. for 48-72 hours or until reoxidized as demonstrated by analytical HPLC, then dialyzed against 3.5 L 50 mM borate pH 8.1 at 4° C. The material was subjected to preparative reverse phase chromatography using a Vydac C-18 column (Hewlett Packard, Wilmington, Del.) or Poros-R2 (PerSeptive Biosystems), heated to 40° C. The column was eluted with (A) 98% water/0.1% TFA, and (B) 100% CH₃CN/0.09% TFA, over 28 minutes, with a flow rate at 1 ml/minute resulting in a final purified product.

Four desalted, purified samples of GAD-β1-α1 were independently infused into a triple quadrapole electrospray mass spectrometer in order to measure the mass of the intact recombinant protein. The average mass obtained from these four measurements was 24434.67+/-2.72 Da. The mass obtained is in excellent agreement with the mass expected from the cDNA-translated sequence, 24432.89 Da. The percent error for the measurement is 0.007% and is typical of the error associated with this type of mass analysis.

In addition, a sample of desalted, purified GAD-β1-α1 was subjected to proteolysis with trypsin to carry out peptide mapping of the protein. The resulting digest was analyzed using MALDI-TOF mass spectrometer. The analysis confirms the presence of a disulfide bridge between Cys50 and Cys112, as one would expect in the properly folded molecule. Additionally, N-terminal sequence analysis confirmed the expected sequence and removal of the Met.

### Example 6

### Protocol for Isolation and Propagation of GAD Reactive Human T Cell Clones and Lines

I. Isolation of Responder Cell Populations

Peripheral blood mononuclear cells (PBMNC), from prediabetic or new onset diabetic patents which should have a source of autoreactive T-cells, were isolated by density centrifugation on ficoll-hypaque. Cells were washed several times and resuspended in 15% PHS Medium (RPMI-1640, 15% heat inactivated normal male pooled human serum (from normal, non-transfused male donors, tested positive in a mixed lymphocyte culture using established techniques), 2 mM L-glutamine, and 5×10⁻⁵ M beta-mercaptoethanol). A portion of the PBMNCs were saved to be used as antigen pulsed antigen presenting cells APCs (see below under stimulators), and a portion frozen for subsequent rounds of stimulation. The remainder were plated on tissue culture plates and incubated for 1 hour at 37° C. to remove adherent cells. The non-adherent cells were removed with the media from the plate and added to a new plate, incubated overnight at 37° C., 5% CO₂ to remove any remaining adherent cell populations.

A non-adherent cell population was harvested and enriched for T cells by passing cells over nylon wool, which removes remaining monocytes and B cells. The cells which did not adhere were enriched for T cells and natural killer cells, by removing CD56+ and CD8+ cells. This was done by collecting the non-adherent cells (depleted of CD56+ and CD8+) by sequential incubation of cells on anti-CD8 antibody coated plates and anti-CD56 antibody coated plates.

II. Preparation of Stimulator Cell Populations; Day 0

PBMNC were incubated in a 0.5 ml volume of 15% PHS media overnight at 37° C., 5% CO₂ with a 1:20 of GAD65 (approximately 50 mg/ml). This can also be achieved using frozen cells which were thawed, washed 2x and incubated with GAD65 for 5-7 hours. The cells were irradiated with 3000 rads, washed 2× and counted.

III. Stimulation of T Cells

1-2×10⁶ CD4+ enriched T cells or Nylon wool enriched T cells or PBL were mixed with 1-2×10⁶ irradiated stimulators, pulsed with no antigen or with whole GAD, in 1.5 ml of 15% PHS medium. After 6 days, 100 μl of the cells were transfered from all conditions of stimulation to two individual wells of a 96 well plate. One microcurie of 3H-thymidine was added to each well for 5 hours and harvested to determine proliferative response of each responder cell population to stimulators pulsed with GAD as compaired to stimulators pulsed with no antigen. On day 7 cells were frozen, or harvested. Harvested cells were washed 2× and restimulated with 1-2×10⁶ stimulators which were prepared as described in II, using fresh or frozen autologous or non-autologous HLA-matched PBMNCs.

10 U/ml human recombinant IL-2 (Research and Development Systems, Minneapolis, Minn.) was added to cultures on Day 8 and Day 11. Cultures were expanded as needed with medium, dividing 1:2 or 1:3 to keep cells at <8×10⁵ cells/ml. Additional IL-2 was added if cells were dividing too quickly and were in need of exogenous IL-2. On day 14, cells are restimulated, as above, to maintain the T cell line, and frozen stocks were created. T cell clones and lines can be created by limiting dilution stimulating with antigen as described above, or cells can be tested for prptide and MHC reaction as described below.

IV. Cloning of T Cells

On day 14, T-cells were harvested, washed, resuspended in 15% PHS medium with 10 U/ml IL-2, and plated with 1×10⁴ stimulators (as prepared above) in terasaki plates (Research and Development Systems) in 15 ml total volume. Cloning can alternatively be started on day 7.

Cells were inspected for growth and transferred to wells, with the cell volume being about ½ of the well volume of a 96 well round bottom plate, in 200 ml 15% PHS medium containing 1×10⁵ stimulators. An additional aliquot of IL-2, to a final concentration of 10 U/ml of 15% PHS medium, was added to the cultures 24 hours later.

As cells grew in the wells, they were tested for antigen reactivity on days 4 or 5, and were split 1:2 into additional wells containing 10 U/ml 15% PHS medium as the cells become confluent.

Cells stocks were frozen from 96 well cultures or were expanded into 24 well, 1.5 ml cultures using T cells from 1 or several of the above wells and 1.5×10⁶ stimulators.

V. Testing Reactivity to GAD

T-cell clones were rested (not given IL-2 for 2 days, at least 7 days post-stimulation with antigen), washed, counted and resuspended in 15% PHS medium. They were plated at 25,000 cells/well in 100 ml 15% PHS medium. Autologous or HLA-class II-matched PBMNCs are loaded with GAD by incubating with GAD (about 50 mg/ml) for at least 5 hours. The cells are washed and irradiated with 3000 rads. These cells are washed and resuspended in 15% PHS medium, and added to the T-cells at a concentration of 1×10⁶ cells/well in 100 ml 15% PHS medium. The cells were incubated for 48 hours, then pulsed with 1 mCi 3H-thymidine and harvested. A positive response is considered to be a stimulation index >3 (stimulation index SI=average cpm of sample stimulated with antigen/average cpm of sample of cells stimulated with no antigen or control antigen). Some controls include T-cells alone, stimulators alone, a purified negative antigen, GAD purified from baculovirus, PHA, and IL-2.

Other methods, well known in the art, for testing clones and lines include dose response to antigen; response to these antigens or negative antigen controls; determination of HLA-class II restriction by adding blocking anti-HLA class II antibody to plates; and use of peptides to load stimulators to determine peptide specificity, which can be done as described above except the peptides are tested by dose titration and left in the assay. A dose response in combination with peptide specificity tests can also be done.

Antigen presenting cells used to determine HLA-restriction include autologous and non-autologous PMNBCs which may have matches and mismatches at the HLA locus and genetically engineered antigen presenting cells to include BLS-1 and mouse L cells or other APCs which expressed only one HLA Class II molecule.

VI. Testing Reactivity to synthetic GAD Peptides

Four individual T cell lines derived from one HLA-DRB1*0404 patient (ThHo) were used to map the 74 synthetic GAD peptides, overlapping sets of 20 mers, that span the entire length of GAD 65 (SEQ. ID. NO. 59). Antigen presenting cells, BLS-DRB1*0404 and/or BLS-DRB1*0401 (Kovats et al., J. Exp. Med. 179:2017-22, 1994), were loaded with peptide by incubating with peptide (about 50 mg/ml) for at least 5 hours. Reactivity of T-cells was determined as above. One peptide, hGAD 33 (PGGAISNMYAMMIARFKMFP SEQ. ID. NO. 40) stimulated 3 or the 4 lines with BLS-B1*0404. COOH terminal truncations of this peptide from 20 amino acids to an 11 amino acid fragment (PGGAISNMYAM SEQ. ID. NO. 39) when presented by either BLS-B1*0404 or BLS-DRB1*0401, stimulated only one of the T-cell lines. A 10 amino acid fragment (PGGAISNMYA SEQ. ID. NO. 41) stimulated the same T-cell line only when presented by BLS-B1*0404. This methodology quickly identifies peptide and HLA restriction of T-cell lines and clones as well as identifying GAD epitopes which stimulate T-cell lines derived from a prediabetic donor.

### Example 7

### Synthesis of GAD Peptides

Peptides amidated at the C terminus were synthesized by solid phase peptide synthesis (SPPS) using Fmoc chemistry. Chemicals used in the synthesis were obtained from Nova Biochem (La Jolla, Calif.). The peptide was assembled on Rink amide MBHA resin (0.25 millimolar scale) starting from the C terminal end by using a 432A Applied Biosystems, Inc. (Foster City, Calif.) automated peptide synthesizer and solid phase strategy. The synthesis required double coupling to ensure completion of the coupling reaction, and HBtu-HOBt coupling chemistry was used. Bolded residues required at least double coupling (SRLSKVAPVIKARMMEYGTT-NH2 (SEQ ID NO:59). Each cycle included Fmoc deprotection of amine from the amino acid residue on the resin, and coupling of incoming Fmoc-amino acid. After successful assembly of the peptide, the resin was washed with dichloromethane and dried under vacuum for two hours. The peptide resin was resuspended in 10 ml trifluoroacetic acid (TFA) containing 1 ml of 4-methoxybenzenethiol and 0.7 g of 4-methylmercaptophenol as scavengers. This suspension was gently mixed at room temperature for 2 hours, then filtered through a PTFE filter, and the filtrate was collected in a capped glass bottle containing 1 liter organic solvent mixture (pentane:acetone=4:1). The white precipitate was allowed to settle at room temperature for 1-2 hours, after which the crude precipitated peptide was isolated by cacantation centrifugation. The crude peptide was washed three times with the organic solvent mixture and dried under vacuum overnight.

Reverse phase HPLC of the crude peptide showed a main peak and smaller impurities which may be deletion peptides. The main peak was isolated by preparative reverse phase HPLC using a solvent gradient consisting of starting buffer A (0.1% TFA) and ending buffer B (70% acetonitrile in 0.1 TFA). Fractions were collected (10-15 ml) and lyophillized to remove all solvent. Fractions were analyzed by reverse HPLC and the pure fractions were further characterized by mass spectrometry.

Peptides having a carboxylic group at the last amino acid at the C-terminus were prepared using solid phase Fmoc chemistry. Peptides were assembled on Wang resin starting from the C-terminal end by using a 431A Applied Biosystems automated peptide synthesizer. Wang resin with the first amino acid attached (Fmoc-Thr(tBu)-Wang) was loaded in the synthesizer, and the couplings were done from the next amino acid at the C-terminus. Double couplings, on those amino acids as indicated above, were done to ensure completion of the coupling reaction. HBtu-HOBt coupling chemistry was used for this purpose. Each cycle included Fmoc deprotection of amine from the amino acid residue on the resin and coupling of incoming Fmoc-amino acid. After successful assembly of the peptide, the resin was washed with dichloromethane and dried for two hours. Cleavage and purification of the peptide is as described above.

Relative affinity of all synthesized peptides for MHC was tested using the DELFIA assay, and engagement of T-cells by peptide:MHC complexes was measured using CTLL cell proliferation in response to IL-2 production by C-terminal amidated GAD65-restricted T-cell hybridomas, as described in later Examples.

### Example 8

### Synthesis of Ala Scan Peptides

A series of 20 C-terminal amidated GAD65 peptides, encompassing amino acids 524 to 543, were synthesized with a single alanine substituted for each non-alanine residue, and a tyrosine was substituted for residues where alanine occurred naturally. The peptides were synthesized by solid phase peptide synthesis (SPPS) strategy by using ABIMED-Gilson AMS 422 multiple peptide synthesizer (Middleton, Wis.). The synthesizer consisted of a Gilson auto-sampler which is capable of X-Y-Z movements, a 48 column reactor module, and amino acid and activating reagent reservoirs. While the reagents and solvents were added to each column by a micro-injector sequentially, the washing of resin in all reaction columns was performed simultaneously.

The peptides were simultaneously assembled and synthesized on the AMS-422 at a 0.025 millimole scale using Rink amide MBHA resin with a substitution of 0.55 millimoles per gram. Twenty columns were set up on the synthesizer with 0.025 millimoles of activated resin in each column. The first step included the removal of Fmoc, which was achieved by using 20% pipiridine in dimethyl formamide (DMF). This operation was simultaneously done on the resin in each reaction column. A sequential mixing protocol was introduced (Thong Luu, Pham Son and Shrikant Deshpande, Automated Multiple Peptide Synthesis:Improvements in Obtaining Quality Peptides, Int. J. Peptides & Proteins, 1995, in press) to maximize the deprotection. A double deprotection strategy was also used to obtain complete deprotection of Fmoc groups. The resin washing step was done simultaneously using DMF.

The first amino acid coupling was achieved by introducing a particular amino acid, activated with pyBOP/HOBt/N-methyl morpholine in DMF (ratio of active sites on the resin to the activated amino acid=1:6), to the designated reaction column by autoinjector. The resin was mixed by a slow bubbling of nitrogen in the reaction column for 20 seconds. Dichloromethane (DCM) was added to the reaction mixture so that the ratio of DMF:DCM was 3:1. The resin was mixed again before another amino acid coupling was initiated in another reaction column. The most hydrophobic amino acids were coupled first so that coupling time is maximum for these amino acids. After the first amino acid was coupled, all the reaction columns were subjected to simultaneous washing with DMF. A double coupling strategy was routinely used in order to complete the amino acid coupling to the resin. After the double coupling was complete, the resin was washed with DMF and the next cycle of Fmoc deprotection and amino acid coupling was activated.

After the final Fmoc deprotection, the peptide resins were washed with DCM and dried. in the reaction columns by applying vacuum on the synthesizer. Columns were removed from the synthesizer and capped at one end using syringe caps (#3980025, Gilson). One and one half milliliters of TFA containing 0.07 g of 4-(methylmercapto)phenol, and 0.1 ml of 4-methoxybenzenethiol, was added to each column, followed by mixing at room temperature for 2 hours. Upon completion of cleavage, the caps at one end of reaction columns were removed, and the reaction mixture was filtered and the filtrate was collected into 100 ml of pentane:acetone (4:1). The peptides were allowed to precipitate for 2 hours at room temperature, and were subsequently isolated by decantation and centrifugation. The pellets were washed three times with pentane:acetone and twice with pentane. The crude peptides were dried in vacuum for 2 hours then subjected to analytical reverse phase-HPLC and mass spectrometry. Those peptides which did not precipitate from the pentane:acetone solution within the 2 hours were cooled to −20° C. overnight, after which they were isolated and washed as above.

Example 9

### Synthesis of Truncated C-Terminal Amidated GAD65 Peptides

A series of C-terminal amidated GAD 65 (SEQ. ID. NO. 59) peptides were synthesized where one or more N-terminal or C-terminal amino acids were systematically truncated (Table 3).

TABLE 3
Truncated GAD65 peptides from amino acid 524 (1) to
amino acid 543 (20). All peptides are amidated at the C-terminus.

| 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|
| S | R | L | S | K | V | A | P | V | I | K | A | R | M | M | E | Y | G | T | T |
| | R | L | S | K | V | A | P | V | I | K | A | R | M | M | E | Y | G | T | T |
| | | L | S | K | V | A | P | V | I | K | A | R | M | M | E | Y | G | T | T |
| | | | S | K | V | A | P | V | I | K | A | R | M | M | E | Y | G | T | T |
| | | | | K | V | A | P | V | I | K | A | R | M | M | E | Y | G | T | T |
| | | | | | V | A | P | V | I | K | A | R | M | M | E | Y | G | T | T |
| | | | | | | A | P | V | I | K | A | R | M | M | E | Y | G | T | T |
| | | | | | | | P | V | I | K | A | R | M | M | E | Y | G | T | T |
| | | | | | | | | V | I | K | A | R | M | M | E | Y | G | T | T |
| | | | | | | | | | I | K | A | R | M | M | E | Y | G | T | T |
| | | | | | | | | | | K | A | R | M | M | E | Y | G | T | T |
| | S | L | S | K | V | A | P | V | I | K | A | R | M | M | E | Y | G | T | |
| | S | L | S | K | V | A | P | V | I | K | A | R | M | M | E | Y | G | | |
| | S | L | S | K | V | A | P | V | I | K | A | R | M | M | E | Y | | | |
| | S | L | S | K | V | A | P | V | I | K | A | R | M | M | E | | | | |
| | S | L | S | K | V | A | P | V | I | K | A | R | M | M | | | | | |
| | S | L | S | K | V | A | P | V | I | K | A | R | M | | | | | | |
| | S | L | S | K | V | A | P | V | I | K | A | R | | | | | | | |
| | S | L | S | K | V | A | P | V | I | K | A | | | | | | | | |
| | S | L | S | K | V | A | P | V | I | K | | | | | | | | | |
| | S | L | S | K | V | A | P | V | I | | | | | | | | | | |

The peptides were synthesized by solid phase peptide synthesis by using an ABIMED-Gilson AMS 422 multiple peptide synthesizer, as described in Example 8.

Example 10

### Truncated C-Terminal Amidated GAD65 Core Peptides

Testing the truncated C-terminal amidated GAD65 peptides of Example 9 showed that the C-terminal truncated peptide (which included amino acids 528 to 543) and the N-terminal truncated peptide (which included amino acids 524 to 539) were still able to bind to I-Ag⁷, and that peptides which included amino acids 528 to 539 were also able to stimulate C-terminal amidated GAD65 peptide restricted T cell hybridomas. Based on this information, a second series of truncated peptides was synthesized based on this core sequence (Table 4), and can be analyzed for MHC affinity and engagement of C-terminal amidated GAD65 restricted T-cell hybridomas.

TABLE 4
Truncated GAD65 core peptides. The C-terminus of
each peptide is amidated. 1 is amino acid 524, 20 is amino acid 543.

| 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|
| | | | S | K | V | A | P | V | I | K | A | R | M | M | E | | | | |
| | | | | K | V | A | P | V | I | K | A | R | M | M | E | | | | |
| | | | | | V | A | P | V | I | K | A | R | M | M | E | | | | |
| | | | | | | A | P | V | I | K | A | R | M | M | E | | | | |
| | | | | | | | P | V | I | K | A | R | M | M | E | | | | |
| | | | S | K | V | A | P | V | I | K | A | R | M | M | | | | | |
| | | | S | K | V | A | P | V | I | K | A | R | M | | | | | | |
| | | | S | K | V | A | P | V | I | K | A | R | | | | | | | |
| | | | S | K | V | A | P | V | I | K | A | | | | | | | | |
| | R | L | S | K | V | A | P | V | I | K | A | R | M | M | E | Y | G | | |
| | R | L | S | K | V | A | P | V | I | K | A | R | M | M | E | Y | | | |
| | R | L | S | K | V | A | P | V | I | K | A | R | M | M | E | | | | |
| | | L | S | K | V | A | P | V | I | K | A | R | M | M | E | Y | G | | |
| | | L | S | K | V | A | P | V | I | K | A | R | M | M | E | Y | | | |
| | | L | S | K | V | A | P | V | I | K | A | R | M | M | E | | | | |
| | | | S | K | V | A | P | V | I | K | A | R | M | M | E | Y | G | | |
| | | | S | K | V | A | P | V | I | K | A | R | M | M | E | Y | | | |
| | | | S | K | V | A | P | V | I | K | | | | | | | | | |

The peptides were synthesized by solid phase peptide synthesis on a 433 A Applied Biosystems automated peptide synthesizer. The peptides were assembled from the carboxy terminal end at 0.05 millimole scale on Rink amide MBHA resin (substitution level 0.55 millimoles per gram). HOBt/HBTU coupling strategy was used for acylation of amines on the resin, and piperdine was used for the deprotection of Fmoc-protected a-amine of the amino acid on the resin. N-methylpyrrolidinone (NMP) was used as the solvent for coupling/deprotection reactions, and dichloromethane (DCM) was used for the final washing of the peptide resin. The deprotection was monitored by measuring the conductivity of Fmoc released. If the deprotection was difficult, the coupling was also difficult, and therefore double coupling and/or acetylation after coupling was introduced into the synthesis.

After assembly of the peptide chain on the resin, the peptide resin was dried under vacuum for 2 hours and subjected to a deprotection protocol. The resin was suspended in 2 ml of trifluoroacetic acid (TFA) containing 0.14 g of 4-methylmercaptophenol and 0.2 ml of 4-methoxybenzenethiol. The suspension was mixed for 2 hours and then filtered into 200 ml of organic solvent (pentane:acetone 4:1). The fine peptide suspension was incubated at −20° C. overnight. The fine suspension had settled, and a film of peptide on the inner surface of the glass bottle was observed. The clear solvent was removed by decantation and the film gently washed with 50 ml of the pentane:acetone mix. The washes were repeated for a total of three washes, followed by two 50 ml washes in pentane. The film was dissolved in 10 ml of 70% aqueous acetonitrile containing 0.1% TFA, and the solution diluted to 30 ml using distilled water. The peptide solution was lyophilized and the resulting white powder characterized by reverse phase HPLC and mass-spectrometry. This product was used for peptide binding and T cell activation assays without further purification.

### Example 11

### Creation of C-Terminal Amidated GAD65 (aa524-543) Restricted Hybridoma T Cell Lines

NOD mouse hybridoma cell lines that express T cell receptors specific to the C-terminal amidated GAD65 peptide have been created. The procedure for obtaining these hybridomas was derived from “Production of Mouse T Cell Hybridomas” in Current Protocols in Immunology, Wiley Interscience, Greene , which is incorporated herein by reference. Briefly, three nine-week old female NOD mice were injected in the foot pads with 50 μg C-terminal amidated GAD65 peptide in 100 ml CFA (Complete Freund's Adjuvant) to cause proliferation of T cells restricted to this peptide. Mice were sacrificed by cervical dislocation eight days later, and the spleen and lymph nodes (popliteal, superficial inguinal) were removed. Lymph nodes were teased between two glass slides into a suspension in Falcon 3002 petri dishes. Spleens were ground into a cell suspension in separate dishes, and then spun at 12,000 RPM for 5 minutes at room temperature. Supernatant was removed, and splenocytes were cleared of red blood cells by lysis: Splenocytes were resuspended in 0.9 ml sterile H₂O for about 5-10 seconds after which 0.1 ml 10× PBS was quickly added followed by approximately 4 ml Bruff's medium (Click's Medium EHAA; Irvine Scientific, Santa Ana, Calif.), 200 ml penicillin/streptomycin (BioWhittaker, Walkersville, Md.), 200 ml L-glutamine (L-Glut, BioWhittaker), 15 g sodium bicarbonate (Sigma, St. Louis, Mo.), 43 ml β-mercaptoethanol (Sigma), 11.6 ml gentamycin sulfate solution (Irvine Scientific), 10 l sterile water) containing 10% fetal bovine serum (FBS, Hyclone, Logan, Utah). The cells were resuspended using a 5 ml pipette, lipid material filtered and discarded. Cells were counted and brought to a concentration of 2×10⁶ cells/ml, and then stimulated in vitro with C-terminal amidated GAD65 peptide at a concentration of 10 mg/ml. Once cells were blasting (approximately 3-5 days), lymphocytes and splenocytes were harvested from culture. Dead cells were removed by centrifugation through Ficoll-Hypaque. Cells were brought to a density of 5×10⁶ to 2×10⁷, and overlaid with Ficoll-Hypaque at a 5 ml to 5 ml ratio. The cells were then centrifuged at 2000 RPM at 4° C., for 20 minutes followed by 2 washes in Bruff's medium with the final wash in Bruff's medium containing 0% FBS. BW5147 cells, a lymphoma cell line (ATCC, Tumor Immunology Bank 48), were harvested and washed in wash medium. BW5147 cells were combined with the splenocytes and lymphocytes in a 1:1 ratio in Bruff's medium containing 20% FBS. The cell mixture was centrifuged for 5 minutes at 2000 RPM, room temperature. The supernatant was aspirated and 1 ml media prewarmed to 37° C. was added. 50% polyethylene glycol (PEG) solution (Sigma) was added to the cell pellet drop-wise over a period of 1 minute to promote cell fusion. The pellet was gently stirred after each drop and then was stirred for one additional minute. Two milliliters of prewarmed wash medium was added drop-wise to the PEG/cell mixture with a 2 ml pipette over a period of 2 minutes, with gentle stirring after each drop. The mixture was then centrifuged for 5 minutes at 2000 RPM and the supernatant discarded. Thymuses from un-primed NOD mice were removed and ground in Bruff's medium containing 20% FBS. The thymocytes were counted and brought to a concentration of 5 x 10⁶ cells/ml. The number of thymocytes to be added was calculated such that splenocytes would be at a number of 0.1−1×10⁵ cells/well with 100 ml/well. This number of thymocytes in Bruff's medium containing 20% FBS was forcefully discharged onto the cell pellet. The cell mixture was then plated on to 96 well plates, 100 ml/well, leaving the outer most wells empty to ensure sterility. The plates were incubated at 37° C., 7.5% CO₂. The next day, 100 ml 2× HAT (Sigma) in Bruff's medium containing 20% FBS was added to each well, and the plate returned to the incubator. On the following days, cells were observed for the death of fusions of two lymphocytes. Only fusions between a lymphoma and a lymphocyte should survive. On day six, 100 ml 2× HAT (Sigma) in Bruff's medium containing 10% FBS was added to each well. On the following days, cells were checked for expansion. Those cells which appeared to be expanding were transferred to a 24 well plate in 1 ml 10× HAT (Sigma) in Bruff's medium containing 20% FBS. Duplicate sets were created and checked daily. Those which were growing were transferred to T-25 flasks. These T-cell hybridomas were gradually weaned to Bruff's medium containing 20% FBS and 0% HAT and maintained for a time until screened for specificity to the C-terminal amidated GAD65 peptide

### Example 12

### Screening C-Terminal Amidated GAD65 Restricted T-Cell Hybridoma Cell Lines

To determine specificity of the T-cell hybridomas, antigen-presenting cells (APCs) were prepared by grinding NOD mice spleens and lysing as in Example 11. The splenocytes were brought to 3 ml in Bruff's medium containing 10% FBS. Mitomycin C (Sigma) was added at 0.3 ml per 3 ml of cell suspension to prevent DNA synthesis. The APCs were incubated for 30 minutes in a 37° C. water bath, and then washed 3 times in Bruff's medium containing 10% FBS, each time centrifuging for 5 minutes at 1200 RPM. After the final wash, the APCs were brought to a concentration of 2×10⁶ cells/ml in Bruff's medium containing 10% FBS. C-terminal amidated GAD65 peptide was titered from 333 μg/ml to 0.15 μg/ml in round bottom 96 well plates. Fifty microliters (1×10⁵) APCs were added to the peptides. Hybridomas were counted and brought to a concentration of 1×10⁶ cells/ml in Bruff's medium containing 10% FBS, and 100 μl (1×10⁵) cells was added to each well. Hybridomas were also tested against the following: I-A^{g7} MHC +a peptide other than C-terminal amidated GAD65 ; an MHC other than I-A^{g7}+C-terminal amidated GAD65 ; the I-A^{g7} MHC alone; and C-terminal amidated GAD65 alone. The plate was incubated at 37° C., 5% CO₂, overnight. The following day, 150 μl of spent medium was removed from each well and transferred to flat bottom 96 well plates and frozen to kill any living cells. Only the spent medium from wells where T cells were activated will contain IL-2. CTLL cells (ATCC TIB-214), which are dependent upon IL-2 for survival, were spun down and washed 3 times in Bruff's medium containing 10% FBS, and plated at a concentration of 5×10³ cells in 50 μl medium in flat bottom 96 well plates. Supernatant collected from the APC/hybridomas was thawed and 50 μl of supernatant was added to the analogous well containing CTLL cells. Two rows were plated as a control for the CTLL cells. Duplicate control wells contained medium and cells alone, or cells, medium and titered IL-2. Plates were incubated at 37° C., 5% CO₂, overnight. The following day the cells were pulsed with ³H-thymidine at 1 μCi/well. Plates were incubated overnight to allow incorporation of ³H-thymidine into the cells. The following day, the cells were harvested in a Skatron Basic 96 Cell Harvester (Carlsbad, Calif.) following the manufacturer's directions. Filtermats were allowed to dry overnight and then placed into sample bags. Approximately 10 ml Beta Scint scintillation fluid (Wallac, Turku, Finland) was added and the bag sealed. Incorporation of ³H-thymidine into the DNA was measured on a Wallac 1205 Betaplate Beta Counter (Turku, Finland). Incorporation of ³H-thymidine by CTLL cells indicates that there was IL-2 in the spent medium, and that the hybridomas originally in that medium had been activated by the C-terminal amidated GAD65 peptide+I-A^{g7} MHC of NOD-derived APCs. Therefore, those wells containing CTLL cells which showed a high proliferative response correspond to hybridomas specific to the peptide:MHC complex. The initial fusion resulted in a hybridoma, MBD.1, which showed a strong proliferative response, >5000 cpm incorporated 3H-thymidine, indicating it is specific to the C-terminal amidated GAD65 peptide+I-A^{g7}. It also had a lesser response >2000 CMP to the same GAD65 peptide lacking C-terminal amidation, but no response to any of the other MHC/peptide combinations. All other cells had stimulation responses of <500 cpm. A second fusion resulted in several additional hybridomas which showed specificity for the C-terminal amidated GAD65 peptide+I-A^{g7} MHC, and these were designated MBD2.3, MBD2.7, MBD2.8, MBD2.11 and MBD2.14.

### Example 13

### Identification of Amino Acid Residues Required for Binding of Peptide to the C-Terminal Amidated GAD65+NOD MHC Class II I-A^{g7} Restricted T Cell Hybridomas

The C-terminal amidated GAD65+I-A^{g7} specific hybridomas described above (MBD.1, MBD2.3, MBD2.7, MBD2.8, MBD2.11 and MBD2.14) were screened for specificity for I-A^{g7}+Ala scan peptides or truncated peptides, using methods described in Example 12. Briefly, the Ala scan peptides or truncated peptides were tested at a series of concentrations between 333 and 0.15 μg/ml. Proliferation of CTLL cells indicated that a particular alanine substitution (or truncation of a particular amino acid) had not affected binding of the MHC-peptide complex to the T cell receptor of a specific hybridoma. Lack of proliferation indicated that the substituted (or truncated) residue was relevant to the binding of the complex by the T cell receptor. Proliferation was severely affected by a single substitution of alanine at amino acid position 524, 526, 527, 528, 529, 531, 532, or 533, or a tyrosine substitution at position 530 or 535, when compared to the unsubstituted control peptide. Activation of T cell hybridomas was seen with truncated peptides which contained amino acids 527-539, with at least one T cell hybridoma recognizing the peptide containing amino acids 529-539, indicating that these residues are critical for binding to the T cell hybridomas tested.

### Example 14

### Peptide Binding to NOD MHC Class II I-A^{g7}

The relative affinity of a given peptide (Ala scan or truncated) for MHC was measured by a Europium-streptavidin dissociation enhanced lanthanide fluoroimmunoassay (DELFIA), as developed by Jensen et al., J. Immunol. Meth. 163:209, 1993. This assay can be used with either whole cells or solublized MHC molecules. Each peptide was assayed in triplicate. In the case of Ala scan peptides, for instance, NOD spleen cells were fixed with 1% paraformaldehyde for 10 minutes at room temperature or 30 minutes on ice, followed by one wash with RPMI 1640, 1% PSN (GIBCO-BRL, Gaithersburg, MD), 200 mM L-glutamine (Hazelton Biologics, Lenexa, Kans.) and 10% heat inactivated fetal calf serum (FCS), and two washes with DPBS (Dulbecco's PBS, BioWhittaker, Walkersville, Md.). Cells were resuspended at 1×10⁷ cells/ml in 0.15 M NaCl containing 1:50 dilutions of protease inhibitor stock solutions D, E, and F (Table 5), 0.01% sodium azide, and 1 M citrate/PO₄, pH 5.5.

### Table 5

### Protease Inhibitor Stock Solutions

Stock D 50×

150 mg phenanthroline

108 mg PMSF (phenylmethylsulfonyl fluoride)

1.8 mg pepstatin

30 mg TPCK (N-Tosyl-L-phenylalanine chloromethyl ketone)

120 mg benzamidine

150 mg iodoacetamide

126 mg NEM

Dissolve in 3 ml methanol.

Stock E 50×

1 mg leupeptin

15 mg TLCK (N-a-p-Tosyl-L-Lysine chloromethyl ketone)

Dissolve in 3 ml H₂0 containing 15 μl of 1M citrate/PO₄, pH 5.5.

Stock F 5OX

8.76 mg EDTA

Dissolve in 3 ml H₂0 containing 15 μl 1 M Tris, pH 8.0.

One hundred microliters of the cell-protease inhibitor mixture was added to each well of a 96-well round-bottom plate (Costar, Pleasanton, Calif.). Fixed NOD cells were co-incubated with biotinylated, C-terminal amidated GAD65 peptide at a concentration of 10,000 nM and unlabeled, Ala scan peptides at concentrations of 100,000, 1,000 and 10 nM for 12-20 hours at 37° C. Mouse serum albumin (MSA), a known allele-specific peptide (SEQ. ID. NO. 61) with high affinity for I-A^{g7}, was used as a positive control, and Eα, which binds to I-A^{d} but not to I-A^{g7}, served as a negative control (Reich et al., J. Immunol. 154:2279-88, 1994). Following incubation, the plates were vortexed and centrifuged in a Beckman GA-6R centrifuge for 10 minutes at 1500 rpm (Beckman, Fullerton, Calif.). The supernatant was removed, and the cells were lysed in 60 μl/well of NP-40 lysis buffer (0.5% NP40, 0.15 M NaCl, 50 mM Tris, pH 8.0, 0.01% sodium azide, and 1:50 dilutions of the protease inhibitor stocks D, E and F (Table 3). The cells were incubated on ice for 30 minutes, with mixing every 15 minutes, followed by centrifuging for 10 minutes at 1500 rpm to obtain a clear lysate.

The assay plates were prepared by coating a 96-well flat bottom plate (Costar) with 100 μl/well anti-I-A^{g7} antibody (10.2.16, 50 μg/ml, TSD Bioservices, Germantown, N.Y.) in DPBS. The plates were incubated for 12-18 hours at 4° C. The unbound antibody was removed and the plate blocked with 200 μl/well MTB (1% BSA, 5% powdered skim milk, 0.01% sodium azide in TTBS (0.1% Tween 20, 0.5 M Tris, 1.5 M NaCl, pH 7.5)) for 30 minutes at room temperature, followed by seven washings in TTBS. Fifty microliters of MTBN (1% BSA, 5% powdered skim milk, 0.01% sodium azide, NP40 in TTBS) was added per well, followed by 50 μl of clear lysate from above. Plates were incubated for 2 hours at 4° C., followed by seven washings with TTBS. Europium-labeled streptavidin (Wallac #1244-360), diluted 1:1000 in DELFIA assay buffer (Table 6), was added to the plate at 100 μl/well.

### Table 6

### DELFIA Assay Buffer

Buffer Stock

0.1 M Tris

0.15 M NaCl

0.05% Sodium azide

0.01% Tween-20

pH 7.75

10 mM DTPA Stock

20 mM Na₂CO₃

DTPA (Diethylenetriaminepentaacetic acid, Sigma, St. Louis, Mo.)

DELFIA Assay Buffer

200 μl 10 mM DTPA stock

100 ml buffer stock

0.5 g BSA (Bovine Serum Albumin)

The plate was incubated for 1 hour at 4° C. followed by seven washings with TTBS. Taking care not to bubble the reagents, 100 μl of Enhancement Solution A (Table 7) was added to each well, and the plate was rocked at room temperature for 3 minutes. Enhancement Solution B (Table 7) was added at 20 μl/well, and the plate rocked for 30 minutes at room temperature. The plate was read on a time-delay fluorometer (Wallac 1234 DELFIA Research Fluorometer).

### Table 7

### Enhancement Solutions A and B

Solution A

2 mM sodium acetate, pH 3.1

0.05% Triton X-100

60 μM BTA (Benzoyl trifluoroacetone, Sigma #B5875)

8.5 μM Yttrium oxide (Sigma #Y3375)

ddH₂O, store at 4° C. in a dark container.

Solution B

250 mM Tris-HCl, pH 7.0

250 Phen (1,10-phenanthroline, Sigma #P1294)

ddH₂O, store at 4° C. in a dark container.

Single substitution of alanine at amino acid position 524, 526, 527, 528, 529, 531, 532, or 533, or substitution of tyrosine at amino acid position 530 or 535, resulted in peptides that were no longer able to compete with unsubstituted, biotinylated C-terminal amidated GAD65 peptide for NOD MHC (I-A^{g7}) binding sites. Substitution of alanine for arginine at position 536 prevented activation in 4 out of the 6 T cell hybridomas. Substitution of alanine for methionine at position 537 prevented activation in 5 out of the 6 hybridomas. Substitution of alanine for methionine at position 538 prevented activation of 1 of the T cell hybridomas. The GAD65 epitope which binds IA^{g7}, as determined by peptide truncation, includes amino acids 527-539. This correlates with the hybridoma data that suggest amino acids 527-539 are involved in binding to the NOD MHC class II molecule, I-Ag⁷ A suitable GAD peptide would be aa 525 to aa 540 (SEQ. ID. NO. 60).

Example 15

In vitro Induction of Anergy With a Peptide-MHC Complex

This assay examines whether a particular peptide-MHC complex will induce anergy in C-terminal amidated GAD65 restricted T cell clones or in in vivo primed lymphocytes.

Flat bottom 96 well plates (Costar) were coated with 100 μl/well (5 μg of antibody/well) anti-class II antibody (10.2.16, 50 μg/ml, TSD Bioservices, Germantown, N.Y.) in DPBS and incubated at 4° C. for 12-18 hours. Unbound antibody was removed and the plates blocked with 5% BSA (bovine serum albumin, Sigma), incubated for 30 minutes at room temperature, followed by 5 to 7 washings in Bruff's medium containing 10% FBS. Peptide-MHC complex, preferably I-A^{g7} complexed with C-terminal amidated GAD65 , or an Ala scan or truncated GAD peptide, was added at 2 and 10 μg/ml. Controls can include peptide-MHC complexes, such as I-A^{g7}-MSA-OH; medium alone; peptide alone, or MHC alone; each of which can be added at the equivalent concentrations as the peptide-MHC complex. The plates were then incubated for 8-10 hours at 4° C. C-terminal amidated GAD65-restricted T cell clones were counted and diluted in Bruff's medium containing 10% FBS so that 6×105 cells were plated per well in 200 μl medium. The plates were incubated at 37° C. for 12-18 hours.

In vivo primed lymphocytes can also be used in place of T cell clones. Briefly, NOD mice were primed with 30-50 μg peptide/150 μl Complete Freund's Adjuvant in the footpad, as described in Example 11. Eight days later the mice were sacrificed, and the spleen, popliteal and supraficial inguinal nodes removed. Tissue was ground, prepared, and Mitomycin C treated, as in Example 11, and was then ready to incorporate into the assay.

The following day, the plates were washed to remove unbound complex, and the cells were pipetted from the plate into separate, labeled Eppendorf tubes, spun at 1200 RPM for 5 minutes, then washed three times with Bruff's medium containing 10% FBS. The cells were counted and each tube was further divided into two tubes, one tube containing ⅓ of the total cell number and the other tube containing the remaining ⅔. The cells were spun again and the tube containing ⅓ of the cells was diluted to 200 μl in Bruff's medium containing 10% FES and 10 U/ml IL-2. The other tube was diluted to 400 μl in Bruff's medium containing 10% FBS, without IL-2.

A second 96-well plate was prepared by adding peptide, such as C-terminal amidated GAD65 at 10 μl/well of 0.6 μg/μl stock, or 0.1 μg/ml anti CD3 (CD3-e cytochrome antibody, Pharmingen, San Diego, Calif.), such that there were at least 2 wells containing α-CD3 and at least 4 wells containing peptide, for each sample to be assayed. Antigen presenting cells (APCs) were prepared as described in Example 12 and diluted to 5×10⁶ cells/ml in Bruff's medium containing 10% FBS, and 100 μl were added only to the wells containing peptide. One hundred microliters of the previously prepared T cell clones or in vivo primed lymphocytes, without IL-2, were added to the wells containing α-CD3 and to half of the wells containing peptide and APCS. Those T cell clones or lymphocytes treated with IL-2 were added only to the remaining wells which contained peptide and APCs, so that the final configuration is such that there were duplicate wells, contain either peptide-MHC complex or control peptide-MHC for each of the three treatments: a-CD3; peptide+APCs with IL-2; and peptide+APCs without IL-2. T cell/lymphocyte concentration should be at least 5×10⁴ cells/well, preferably about 2.3×10⁵ to about 5.3×10⁵. The plates were incubated at 37° C. for 3 days.

The cells were then pulsed with ³H-thymidine at 1 μCi/well. Plates were incubated for 5 hours to allow incorporation of ³H-thymidine into the cellular DNA. The cells were then harvested in a Skatron Basic 96 Cell Harverster following manufacturer's directions. Filtermats were allowed to dry overnight and then placed into sample bags. Approximately 10 ml Beta Scint scintillation fluid (Wallac, Turku, Finland) was added and the bag sealed. Incorporation of ³H-thymidine into the DNA was measured on a Wallac 1205 Betaplate Beta Counter (Turku, Finland) Incorporation of ³H-thymidine by the T-cells indicates that the T-cells were rescued from anergy by the addition of IL-2. If the T-cells were anergized, followed by addition of APCs and peptide (but not IL-2), they should not respond to APCs and peptide, and there should be no incorporation of ³H-thymidine. As a control, a-CD3 was used to show that the cells were indeed alive and responding normally to other stimulators.

### Example 16

### Adoptive transfer

IDDM can be adoptively transferred by injecting splenic cells from a diabetic donor into a non-diabetic recipient. Female NOD/CaJ mice were screened for diabetes by monitoring urinary glucose levels. Those animals showing positive urine values of at least 250 mg/dl glucose were further analyzed for blood glucose levels using tail clippings, and if the blood glucose was also at or above 250 mg/dl, the mice were classified as overtly diabetic.

Newly diabetic NOD mice were irradiated (730 rad) and randomly divided into 4 treatment groups, and splenocytes were isolated as described above. Non-diabetic 7-8 week old, NOD recipient mice were divided into 4 groups. Group one received 1×10⁷ splenocytes, injected intravenously. Six hours following the injection the mice received a second intravenous injection of either saline, 10 μg/mouse C-terminal amidated GAD65 peptide, or 10, 5, or 1 μg/mouse C-terminal amidated GAD65 peptide-MHC complex. Group two received 2×10⁷ splenocytes, followed by injections with either saline, 10 μg/mouse C-terminal amidated GAD65 peptide-MHC complex, or 5 μg/mouse MSA-MHC complex. Group three received 1×10⁷ splenocytes and injections of either saline, 10 μg/mouse C-terminal amidated GAD65 or 200 μg/mouse 10.2.16, an anti-class II antibody. Group four received 1×10⁷ splenocytes followed by injection with either saline, 20 μg/mouse C-terminal amidated GAD65 peptide, or 1, 5 or 10 μg/mouse C-terminal amidated GAD65 peptide-MHC complex. -Group four mice received only two treatments with peptide or peptide-MHC complex, one on day 0 and a second on day 4. All other groups received further treatments on days 8 and 12. The mice were tested for the onset of diabetes by urine analysis. On the day the first animal showed overt signs of diabetes, as determined by urine and blood glucose levels, mice from each of the treatment groups were randomly selected, and urine and blood glucose levels determined for all selected mice, which were then sacrificed, and spleens and pancreases removed for immunohistochemical analysis. Saline-treated mice developed diabetes within about 12-20 days. Group one mice, which received four treatments of 10 μg peptide-MHC complex, had no significant development of disease by day 30, and did not develop disease until day 75. Those receiving 5 μg peptide-MHC complex had stabilized at 40% diseased mice by day 30, with a gradual increase in disease onset up to day 80, when there was 100% disease among the mice. Those mice in group four, which received only two treatments of peptide-MHC complex, experienced some delayed onset of disease, i.e., less than 50% of those mice receiving 10 μg of peptide-MHC had developed disease by day 30. Blocking with anti-MHC antibody in group three delayed the onset of disease, but provided less protection, i.e., over 75% of those mice receiving 10 μg peptide alone had developed disease by day 30. The C-terminal amidated GAD 65 (SEQ. ID. NO. 59) peptide alone accelerated the onset of diabetes in this adoptive transfer model, while the peptide-MHC complex prevented onset of disease.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

EXHIBIT A
TO ASSIGNMENT OF TECHNOLOGY

| | | ZymoGenetics/Novo Nordisk |
|---|---|---|
| Serial No. | Filing Date | Reference # |
| 08/480,002 | 06/07/95 | 95-25 |
| 08/657,581 | 06/07/96 | 95-25-C1 |
| PCT/US96/10102 | 06/07/96 | 95-25-PC |
| 08/483,241 | 06/07/95 | 95-26 |
| 08/855,925 | 05/14/97 | 95-26 con. |
| 08/482,133 | 06/07/95 | 95-27 |
| 60/005,964 | 10/27/95 | 95-30 |

1. A soluble, fused MHC heterodimer:peptide complex comprising:
a first DNA segment encoding at least a portion of a first domain of a selected MHC molecule;
a second DNA segment encoding at least a portion of a second domain of the selected MHC molecule;
a first linker DNA segment encoding about 5 to about 25 amino acids and connecting in-frame the first and second DNA segments;
wherein linkage of the first DNA segment to the second DNA segment by the first linker DNA segment results in a fused first DNA-first linker-second DNA polysegment;
a third DNA segment encoding an antigenic peptide capable of associating with a peptide binding groove of the selected MHC molecule;
a second linker DNA segment encoding about 5 to about 25 amino acids and connecting in-frame the third DNA segment to the fused first DNA-first linker-second DNA polysegment;
wherein linkage of the third DNA segment to the fused first-first linker-second DNA polysegment by the second linker DNA segment results in a soluble, fused MHC heterodimer:peptide complex.

2. The soluble, fused MHC heterodimer:peptide complex of claim 1, wherein the selected MHC molecule is an MHC Class II molecule.

3. The soluble, fused MHC heterodimer:peptide complex of claim 2, wherein the first DNA segment encodes a β1 domain.

4. The soluble, fused MHC heterodimer:peptide complex of claim 2, wherein the second DNA segment encodes an α1 domain or α1α2 domains.

5. The soluble, fused MHC heterodimer:peptide complex of claim 1, wherein the selected MHC molecule is selected from the group consisting of IA^{g7}, IA^{s}, DR1β*1501 and DRA*0101.

6. The soluble, fused MHC heterodimer:peptide complex of claim 1, wherein the selected MHC molecule is an MHC Class I molecule.

7. The soluble, fused MHC heterodimer:peptide complex of claim 1, wherein the first linker DNA segment is GASAG (SEQ. ID. NO. 29) or GGGGSGGGGSGGGGS (SEQ. ID. NO. 36).

8. The soluble, fused MHC heterodimer:peptide complex of claim 1, wherein the second linker DNA segment is GGSGG (SEQ. ID. NO. 30) or GGGSGGS (SEQ. ID. NO. 31).

9. The soluble, fused MHC heterodimer:peptide complex of claim 1, wherein the third DNA segment encodes an antigenic peptide capable of stimulating an MHC-mediated immune response.

10. The antigenic peptide of claim 9, wherein the peptide is selected from the group consisting of a mammalian GAD 65 peptide, (SEQ ID NO: 59), (SEQ. ID. NO. 61), (SEQ ID NO:40), (SEQ. ID. NO. 39) and a mammalian mylein basic peptide(SEQ. ID. NO. 33).

11. The soluble, fused MHC heterodimer:peptide complex of claim 1, wherein said MHC heterodimer:peptide complex further comprises a fourth DNA segment encoding at least a portion of a third domain of the selected MHC molecule, and a third linker DNA segment encoding about 5 to about 25 amino acids and connecting in-frame the second and fourth DNA segments resulting in a fused third DNA-second linker-first DNA-first linker-second DNA-third linker-fourth DNA polysegment.

12. The soluble, fused MHC heterodimer:peptide complex of claim 11, wherein the selected MHC molecule is an MHC Class I molecule.

13. The soluble, fused MHC heterodimer:peptide complex of claim 11, wherein the selected MHC molecule is an MHC Class II molecule.

14. The soluble, fused MHC heterodimer:peptide complex of claim 11, wherein the fourth DNA segment is a β2 chain.

15. The soluble, fused MHC heterodimer:peptide complex of claim 11, wherein the third linker DNA segment is GGGGSGGGGSGGGGSGGGGSGGGGS (SEQ. ID. NO. 32).

16. An isolated polynucleotide molecule encoding a soluble, fused MHC heterodimer:peptide complex of claim 1.

17. A fusion protein expression vector capable of expressing a soluble, fused MHC heterodimer:peptide complex of claim 1, comprising the following operably linked elements:
a transcription promoter;
a first DNA segment encoding at least a portion of a first domain of a selected MHC molecule;
a second DNA segment encoding at least a portion of a second domain of the selected MHC molecule;
a first linker DNA segment encoding about 5 to about 25 amino acids and connecting in-frame the first and second DNA segments;
wherein linkage of the first DNA segment to the second DNA segment by the first linker DNA segment results in a fused first DNA-first linker-second DNA polysegment;
a third DNA segment encoding an antigenic peptide capable of associating with a peptide binding groove of the selected MHC molecule;
a second linker DNA segment encoding about 5 to about 25 amino acids and connecting in-frame the third DNA segment to the fused first DNA-first linker-second DNA polysegment;
wherein linkage of the third DNA segment to the fused first DNA-first linker-second DNA polysegment by the second linker DNA segment results in expression of a soluble, fused MHC heterodimer:peptide complex; and
a transcription terminator.

18. The expression vector of claim 17, wherein said MHC heterodimer:peptide complex further comprises a fourth DNA segment encoding at least a portion of a third domain of the selected MHC molecule, and a third linker DNA segment encoding about 5 to about 25 amino acids and connecting in-frame the second and fourth DNA segments resulting in a fused third DNA-second linker-first DNA-first linker-second DNA-third linker-fourth DNA polysegment.

19. A soluble, fused MHC heterodimer:peptide complex produced by culturing a cell into which has been introduced an expression vector according to claim 17, whereby said cell expresses a soluble, fused MHC heterodimer:peptide complex encoded by the DNA polysegment; and recovering the soluble, fused MHC heterodimer:peptide complex.

20. A pharmaceutical composition comprising a soluble, fused MHC heterodimer:peptide complex of claim 1 in combination with a pharmaceutically acceptable vehicle.

21. An antibody that binds to an epitope of a soluble, fused MHC heterodimer:peptide complex of claim 1.

22. A method of treating a patient to decrease an autoimmune response, the method comprising inducing immunological tolerance in said patient by administering a therapeutically effective amount of a soluble, fused MHC heterodimer:peptide complex of claim 1.

23. A method for preparing a responder cell clone that proliferates when combined with a selected antigenic peptide presented by a stimulator cell, comprising:
isolating non-adherent, CD56-, CD8-cells that are reactive with the selected antigenic peptide, thereby forming responder cells;
stimulating the responder cells with pulsed or primed stimulator cells;
restimulating the stimulated responder cells with pulsed or primed stimulator cells; and
isolating a responder cell clone.

24. The method of claim 23, wherein the responder cells are isolated from a prediabetic or new onset diabetic patient.

25. The method of claim 23, wherein the responder cell clone is a T cell clone.

26. The method of claim 23, wherein the selected antigenic peptide is a GAD peptide.


SEQUENCE LISTING (121 sequences)

SEQ ID NO: 1 - DNA, 33 bp; single-stranded, linear
gcgcaagcttgaattcgagctcatggtgtgtct

SEQ ID NO: 2 - DNA, 58 bp; single-stranded, linear
aattcgatatcatggtgtgtctgaagctccctggaggctcctgcatgacagcgctgac

SEQ ID NO: 3 - DNA, 58 bp; single-stranded, linear
cactgtcagcgctgtcatgcaggagcctccagggagcttcagacacaccatgatatcg

SEQ ID NO: 4 - DNA, 60 bp; single-stranded, linear
acttctttaaaaacatcgtgactccgcgtacacccccgccatcgggaggcgggtcaggtg

SEQ ID NO: 5 - DNA, 60 bp; single-stranded, linear
gatccacctgacccgcctcccgatggcgggggtgtacgcggagtcacgatgtttttaaag

SEQ ID NO: 6 - DNA, 59 bp; single-stranded, linear
agtgacactgatggtgctgagctccccactggctttgtctgacgaaaacccagtagtgc

SEQ ID NO: 7 - DNA, 59 bp; single-stranded, linear
aagtgcactactgggttttcgtcagacaaagccagtggggagctcagcaccatcagtgt

SEQ ID NO: 8 - DNA, 27 bp; single-stranded, linear
gccggctgatgctccccgctgcactgt

SEQ ID NO: 9 - DNA, 25 bp; single-stranded, linear
gcgctctagatcatatagttggagc

SEQ ID NO: 10 - DNA, 37 bp; single-stranded, linear
ccagggtctagatcataaaggccctgggtgtctggag

SEQ ID NO: 11 - DNA, 37 bp; single-stranded, linear
cgaggaattcgcagagacctcccagagaccaggatcc

SEQ ID NO: 12 - DNA, 37 bp; single-stranded, linear
aacactctagatcactgcaggagccctgctggaggag

SEQ ID NO: 13 - DNA, 37 bp; single-stranded, linear
cgaggaattctgagtcctggtgactgccattacctgt

SEQ ID NO: 14 - DNA, 30 bp; single-stranded, linear
ggagcatcagccggcatcaaagaagaacat

SEQ ID NO: 15 - DNA, 111 bp; single-stranded, linear
gaggatgattaaatgagtcgcctctcgaaggtggctccagtgattaaagccagaatgatggagtatggaaccacaggaggtggaggctctggaggtggaggctcaggagga

SEQ ID NO: 16 - DNA, 39 bp; single-stranded, linear
ggaggctcaggaggaggtgggtccggagactccgaaagg

SEQ ID NO: 17 - DNA, 32 bp; single-stranded, linear
cgcgggatccgatcgtggaggatgattaaatg

SEQ ID NO: 18 - DNA, 30 bp; single-stranded, linear
gccacctgatccaccccgcagggaggtggg

SEQ ID NO: 19 - DNA, 30 bp; single-stranded, linear
ggtggatcaggtggcgaagacgacattgag

SEQ ID NO: 20 - DNA, 30 bp; single-stranded, linear
ccggaattcttaactagtagctggggtgaa

SEQ ID NO: 21 - DNA, 30 bp; single-stranded, linear
ccggaattcttaactagtagctggggtgga

SEQ ID NO: 22 - DNA, 30 bp; single-stranded, linear
gccacctgatccaccccgcagggaggtgtg

SEQ ID NO: 23 - DNA, 107 bp; single-stranded, linear
cgcgggatccgatcgtggaggatgattaaatgttctttaaaaacatcgtgactccgcgtacacccccgccaggaggtggaggctctggaggtggaggctcaggagga

SEQ ID NO: 24 - DNA, 30 bp; single-stranded, linear
ccggaattcttacttgctccgggcagactc

SEQ ID NO: 25 - DNA, 72 bp; single-stranded, linear
ggaggctcaggaggaggtgggtctggcggtggaggttccggcggaggcggttcagaagacgacattgaggcc

SEQ ID NO: 26 - DNA, 63 bp; single-stranded, linear
ggcggaggtggctcaggcggaggtggatctggaggtggaggctcacggcttgaacagcccaat

SEQ ID NO: 27 - DNA, 63 bp; single-stranded, linear
tgagcctccacctccagatccacctccgcctgagccacctccgccagtctctgtcagctctga

SEQ ID NO: 28 - DNA, 33 bp; single-stranded, linear
ccggaattcttaactagtctctgtcagctctga

SEQ ID NO: 29 - protein, 5 aa
GASAG

SEQ ID NO: 30 - protein, 5 aa
GGSGG

SEQ ID NO: 31 - protein, 7 aa
GGGSGGS

SEQ ID NO: 32 - protein, 25 aa
GGGGSGGGGSGGGGSGGGGSGGGGS

SEQ ID NO: 33 - protein, 21 aa
DENPVVHFFKNIVTPRTPPPS

SEQ ID NO: 34 - protein, 8 aa
GGSGGGGS

SEQ ID NO: 35 - DNA, 15 bp; single-stranded, linear
ggaggctcaggagga

SEQ ID NO: 36 - protein, 15 aa
GGGGSGGGGSGGGGS

SEQ ID NO: 37 - protein, 13 aa
FFKNIVTPRTPPP

SEQ ID NO: 38 - protein, 12 aa
GGGGSGGGGSGG

SEQ ID NO: 39 - protein, 11 aa
PGGAISNMYAM

SEQ ID NO: 40 - protein, 20 aa
PGGAISNMYAMMIARFKNFP

SEQ ID NO: 41 - protein, 10 aa
PGGAISNMYA

SEQ ID NO: 42 - protein, 4 aa
ERLE

SEQ ID NO: 43 - protein, 5 aa
DPVVH

SEQ ID NO: 44 - protein, 14 aa
FFKNIVTPRTPPPS

SEQ ID NO: 45 - protein, 5 aa
GGGSG

SEQ ID NO: 46 - protein, 8 aa
GGGSGGSG

SEQ ID NO: 47 - protein, 20 aa
SRLSKVAPVIKARMMEYGTT

SEQ ID NO: 48 - DNA, 16 bp; single-stranded, linear
catcgtggaggatgat

SEQ ID NO: 49 - DNA, 15 bp; single-stranded, linear
gaggatgattaaatg

SEQ ID NO: 50 - protein, 20 aa
  Modified-site 20: /product= “OTHER” /note= “Xaa = threoninamide”
SRLSKVAPVIKARMMEYGTX

SEQ ID NO: 51 - protein, 19 aa
  Modified-site 19: /product= “OTHER” /note= “Xaa = threoninamide”
RLSKVAPVIKARMMEYGTX

SEQ ID NO: 52 - protein, 18 aa
  Modified-site 18: /product= “OTHER” /note= “Xaa = threoninamide”
LSKVAPVIKARMMEYGTX

SEQ ID NO: 53 - protein, 17 aa
  Modified-site 17: /product= “OTHER” /note= “Xaa = threoninamide”
SKVAPVIKARMMEYGTX

SEQ ID NO: 54 - protein, 16 aa
  Modified-site 16: /product= “OTHER” /note= “Xaa = threoninamide”
KVAPVIKARMMEYGTX

SEQ ID NO: 55 - protein, 15 aa
  Modified-site 15: /product= “OTHER” /note= “Xaa = threoninamide”
VAPVIKARMMEYGTX

SEQ ID NO: 56 - protein, 14 aa
  Modified-site 14: /product= “OTHER” /note= “Xaa = threoninamide”
APVIKARMMEYGTX

SEQ ID NO: 57 - protein, 13 aa
  Modified-site 13: /product= “OTHER” /note= “Xaa = threoninamide”
PVIKARMMEYGTX

SEQ ID NO: 58 - protein, 12 aa
  Modified-site 12: /product= “OTHER” /note= “Xaa = threoninamide”
VIKARMMEYGTX

SEQ ID NO: 59 - protein, 11 aa
  Modified-site 11: /product= “OTHER” /note= “Xaa = threoninamide”
IKARMMEYGTX

SEQ ID NO: 60 - protein, 10 aa
  Modified-site 10: /product= “OTHER” /note= “Xaa = threoninamide”
KARMMEYGTX

SEQ ID NO: 61 - protein, 18 aa
  Modified-site 18: /product= “OTHER” /note= “Xaa = threoninamide”
SLSKVAPVIKARMMEYGX

SEQ ID NO: 62 - protein, 17 aa
  Modified-site 17: /product= “OTHER” /note= “Xaa = glycinamide”
SLSKVAPVIKARMMEYX

SEQ ID NO: 63 - protein, 16 aa
  Modified-site 16: /product= “OTHER” /note= “Xaa = tyrosinamide”
SLSKVAPVIKARMMEX

SEQ ID NO: 64 - protein, 15 aa
  Modified-site 15: /product= “OTHER” /note= “Xaa = glutamic acid amide”
SLSKVAPVIKARMMX

SEQ ID NO: 65 - protein, 14 aa
  Modified-site 14: /product= “OTHER” /note= “Xaa = methioninamide”
SLSKVAPVIKARMX

SEQ ID NO: 66 - protein, 13 aa
  Modified-site 13: /product= “OTHER” /note= “Xaa = methioninamide”
SLSKVAPVIKARX

SEQ ID NO: 67 - protein, 12 aa
  Modified-site 12: /product= “OTHER” /note= “Xaa = argininamide”
SLSKVAPVIKAX

SEQ ID NO: 68 - protein, 11 aa
  Modified-site 11: /product= “OTHER” /note= “Xaa = alaninamide”
SLSKVAPVIKX

SEQ ID NO: 69 - protein, 10 aa
  Modified-site 10: /product= “OTHER” /note= “Xaa = lysinamide”
SLSKVAPVIX

SEQ ID NO: 70 - protein, 9 aa
  Modified-site 9: /product= “OTHER” /note= “Xaa = isoleucinamide”
SLSKVAPVX

SEQ ID NO: 71 - protein, 13 aa
  Modified-site 13: /product= “OTHER” /note= “Xaa = glutamic acid amide”
SKVAPVIKARMMX

SEQ ID NO: 72 - protein, 12 aa
  Modified-site 12: /product= “OTHER” /note= “Xaa = glutamic acid amide”
KVAPVIKARMMX

SEQ ID NO: 73 - protein, 11 aa
  Modified-site 11: /product= “OTHER” /note= “Xaa = glutamic acid amide”
VAPVIKARMMX

SEQ ID NO: 74 - protein, 10 aa
  Modified-site 10: /product= “OTHER” /note= “Xaa = glutamic acid amide”
APVIKARMMX

SEQ ID NO: 75 - protein, 9 aa
  Modified-site 9: /product= “OTHER” /note= “Xaa = glutamic acid amide”
PVIKARMMX

SEQ ID NO: 76 - protein, 12 aa
  Modified-site 12: /product= “OTHER” /note= “Xaa = methioninamide”
SKVAPVIKARMX

SEQ ID NO: 77 - protein, 11 aa
  Modified-site 11: /product= “OTHER” /note= “Xaa = methioninamide”
SKVAPVIKARX

SEQ ID NO: 78 - protein, 10 aa
  Modified-site 10: /product= “OTHER” /note= “Xaa = argininamide”
SKVAPVIKAX

SEQ ID NO: 79 - protein, 9 aa
  Modified-site 9: /product= “OTHER” /note= “Xaa = alaninamide”
SKVAPVIKX

SEQ ID NO: 80 - protein, 17 aa
  Modified-site 17: /product= “OTHER” /note= “Xaa = glycinamide”
RLSKVAPVIKARMMEYX

SEQ ID NO: 81 - protein, 16 aa
  Modified-site 16: /product= “OTHER” /note= “Xaa = tyrosinamide”
RLSKVAPVIKARMMEX

SEQ ID NO: 82 - protein, 15 aa
  Modified-site 15: /product= “OTHER” /note= “Xaa = glutamic acid amide”
RLSKVAPVIKARMMX

SEQ ID NO: 83 - protein, 16 aa
  Modified-site 16: /product= “OTHER” /note= “Xaa = glycinamide”
LSKVAPVIKARMMEYX

SEQ ID NO: 84 - protein, 15 aa
  Modified-site 15: /product= “OTHER” /note= “Xaa = tyrosinamide”
LSKVAPVIKARMMEX

SEQ ID NO: 85 - protein, 14 aa
  Modified-site 14: /product= “OTHER” /note= “Xaa = glutamic acid amide”
LSKVAPVIKARMMX

SEQ ID NO: 86 - protein, 15 aa
  Modified-site 15: /product= “OTHER” /note= “Xaa = glycinamide”
SKVAPVIKARMMEYX

SEQ ID NO: 87 - protein, 14 aa
  Modified-site 14: /product= “OTHER” /note= “Xaa = tyrosinamide”
SKVAPVIKARMMEX

SEQ ID NO: 88 - protein, 8 aa
  Modified-site 8: /product= “OTHER” /note= “Xaa = lysinamide”
SKVAPVIX

SEQ ID NO: 89 - protein, 15 aa
KPKATAEQLKTVMDD

SEQ ID NO: 90 - DNA, 243 bp; single-stranded, linear
  CDS 1..243
atcaaagaagaacatgtgatcatccaggccgagttctatctgaatcctgaccaatcaggcgaatttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatatgatc

SEQ ID NO: 91 - protein, 81 aa
IKEEHVIIQAEFYLNPDQSGEFMFDFDGDEIFHVDMAKKETVWRLEEFGRFASFEAQGALANIAVDKANLEIMTKRSNYMI

SEQ ID NO: 92 - DNA, 621 bp; single-stranded, linear
  CDS 1..621
gacgaaaacccagtagtgcacttctttaaaaacatcgtgactccgcgtacacccccgccatcgggaggcgggtcaggtggatccggggacacccgaccacgtttcctgtggcagcctaagagggagtgtcatttcttcaatgggacggagcgggtgcggttcctggacagatacttctataaccaggaggagtccgtgcgtttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcggggagcatcagccggcatcaaagaagaacatgtgatcatccaggccgagttctatctgaatcctgaccaatcaggcgaatttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatatgatc

SEQ ID NO: 93 - protein, 207 aa
DENPVVHFFKNIVTPRTPPPSGGGSGGSGDTRPRFLWQPKRECHFFNGTERVRFLDRYFYNQEESVRFDSDVGEFRAVTELGRPDAEYWNSQKDILEQARAAVDTYCRHNYGVVESFTVQRGASAGIKEEHVIIQAEFYLNPDQSGEFMFDFDGDEIFHVDMAKKETVWRLEEFGRFASFEAQGALANIAVDKANLEIMTKRSNYMI

SEQ ID NO: 94 - DNA, 273 bp; single-stranded, linear
  CDS 1..273
ggagactccgaaaggcatttcgtgcaccagttcaagggcgagtgctacttcaccaacgggacgcagcgcatacggctcgtgaccagatacatctacaaccgggaggagtacctgcgcttcgacagcgacgtgggcgagtaccgcgcggtgaccgagctggggcggcactcagccgagtactacaataagcagtacctggagcgaacgcgggccgagctggacacggcgtgcagacacaactacgaggagacggaggtccccacctccctgcgg

SEQ ID NO: 95 - protein, 91 aa
GDSERHFVHQFKGECYFTNGTQRIRLVTRYIYNREEYLRFDSDVGEYRAVTELGRHSAEYYNKQYLERTRAELDTACRHNYEETEVPTSLR

SEQ ID NO: 96 - DNA, 261 bp; single-stranded, linear
  CDS 1..261
gaagacgacattgaggccgaccacgtaggcttctatggtacaactgtttatcagtctcctggagacattggccagtacacacatgaatttgatggtgatgagttgttctatgtggacttggataagaagaaaactgtctggaggcttcctgagtttggccaattgatactctttgagccccaaggtggactgcaaaacatagctgcagaaaaacacaacttgggaatcttgactaagaggtcaaatttcaccccagctact

SEQ ID NO: 97 - protein, 87 aa
EDDIEADHVGFYGTTVYQSPGDIGQYTHEFDGDELFYVDLDKKKTVWRLPEFGQLILFEPQGGLQNIAAEKHNLGILTKRSNFTPAT

SEQ ID NO: 98 - DNA, 654 bp; single-stranded, linear
  CDS 1..654
agtcgcctctcgaaggtggctccagtgattaaagccagaatgatggagtatggaaccacaggaggtggaggctctggaggtggaggctcaggaggaggtgggtccggagactccgaaaggcatttcgtgcaccagttcaagggcgagtgctacttcaccaacgggacgcagcgcatacggctcgtgaccagatacatctacaaccgggaggagtacctgcgcttcgacagcgacgtgggcgagtaccgcgcggtgaccgagctggggcggcactcagccgagtactacaataagcagtacctggagcgaacgcgggccgagctggacacggcgtgcagacacaactacgaggagacggaggtccccacctccctgcggggtggatcaggtggcgaagacgacattgaggccgaccacgtaggcttctatggtacaactgtttatcagtctcctggagacattggccagtacacacatgaatttgatggtgatgagttgttctatgtggacttggataagaagaaaactgtctggaggcttcctgagtttggccaattgatactctttgagccccaaggtggactgcaaaacatagctgcagaaaaacacaacttgggaatcttgactaagaggtcaaatttcaccccagctact

SEQ ID NO: 99 - protein, 218 aa
SRLSKVAPVIKARMMEYGTTGGGGSGGGGSGGGGSGDSERHFVHQFKGECYFTNGTQRIRLVTRYIYNREEYLRFDSDVGEYRAVTELGRHSAEYYNKQYLERTRAELDTACRHNYEETEVPTSLRGGSGGEDDIEADHVGFYGTTVYQSPGDIGQYTHEFDGDELFYVDLDKKKTVWRLPEFGQLILFEPQGGLQNIAAEKHNLGILTKRSNFTPAT

SEQ ID NO: 100 - DNA, 273 bp; single-stranded, linear
  CDS 1..273
ggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcgg

SEQ ID NO: 101 - protein, 91 aa
GDSERHFVFQFKGECYFTNGTQRIRSVDRYIYNREEYLRFDSDVGEYRAVTELGRPDPEYYNKQYLEQTRAELDTVCRHNYEGVETHTSLR

SEQ ID NO: 102 - DNA, 261 bp; single-stranded, linear
  CDS 1..261
gaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctact

SEQ ID NO: 103 - protein, 87 aa
EDDIEADHVGVYGTTVYQSPGDIGQYTHEFDGDEWFYVDLDKKETIWMLPEFGQLTSFDPQGGLQNIATGKYTLGILTKRSNSTPAT

SEQ ID NO: 104 - DNA, 633 bp; single-stranded, linear
  CDS 1..633
ttctttaaaaacatcgtgactccgcgtacacccccgccaggaggtggaggctctggaggtggaggctcaggaggaggtgggtccggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggggtggatcaggtggcgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctact

SEQ ID NO: 105 - protein, 211 aa
FFKNIVTPRTPPPGGGGSGGGGSGGGGSGDSERHFVFQFKGECYFTNGTQRIRSVDRYIYNREEYLRFDSDVGEYRAVTELGRPDPEYYNKQYLEQTRAELDTVCRHNYEGVETHTSLRGGSGGEDDIEADHVGVYGTTVYQSPGDIGQYTHEFDGDEWFYVDLDKKETIWMLPEFGQLTSFDPQGGLQNIATGKYTLGILTKRSNSTPAT

SEQ ID NO: 106 - DNA, 312 bp; single-stranded, linear
  CDS 1..312
cggcttgaacagcccaatgtcgccatctccctgtccaggacagaggccctcaaccaccacaacactctggtctgttcggtgacagatttctacccagccaagatcaaagtgcgctggttcaggaatggccaggaggagacagtgggggtctcatccacacagcttattaggaatggggactggaccttccaggtcctggtcatgctggagatgacccctcatcagggagaggtctacacctgccatgtggagcatcccagcctgaagagccccatcactgtggagtggagggcacagtccgagtctgcccgg

SEQ ID NO: 107 - protein, 104 aa
RLEQPNVAISLSRTEALNHHNTLVCSVTDFYPAKIKVRWFRNGQEETVGVSSTQLIRNGDWTFQVLVMLEMTPHQGEVYTCHVEHPSLKSPITVEWRAQSESAR

SEQ ID NO: 108 - DNA, 588 bp; single-stranded, linear
  CDS 1..588
gaagacgacattgaggccgaccacgtaggcttctatggtacaactgtttatcagtctcctggagacattggccagtacacacatgaatttgatggtgatgagttgttctatgtggacttggataagaagaaaactgtctggaggcttcctgagtttggccaattgatactctttgagccccaaggtggactgcaaaacatagctgcagaaaaacacaacttgggaatcttgactaagaggtcaaatttcaccccagctaccaatgaggctcctcaagcgactgtgttccccaagtcccctgtgctgctgggtcagcccaacacccttatctgctttgtggacaacatcttcccacctgtgatcaacatcacatggctcagaaatagcaagtcagtcacagacggcgtttatgagaccagcttcctcgtcaaccgtgaccattccttccacaagctgtcttatctcaccttcatcccttctgatgatgacatttatgactgcaaggtggagcactggggcctggaggagccggttctgaaacactgggaacctgagattccagcccccatgtcagagctgacagagact

SEQ ID NO: 109 - protein, 196 aa
EDDIEADHVGFYGTTVYQSPGDIGQYTHEFDGDELFYVDLDKKKTVWRLPEFGQLILFEPQGGLQNIAAEKHNLGILTKRSNFTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWEPEIPAPMSELTET

SEQ ID NO: 110 - DNA, 1344 bp; single-stranded, linear
  CDS 1..1344
agtcgcctctcgaaggtggctccagtgattaaagccagaatgatggagtatggaaccacaggaggtggaggctctggaggtggaggctcaggaggaggtgggtccggagactccgaaaggcatttcgtgcaccagttcaagggcgagtgctacttcaccaacgggacgcagcgcatacggctcgtgaccagatacatctacaaccgggaggagtacctgcgcttcgacagcgacgtgggcgagtaccgcgcggtgaccgagctggggcggcactcagccgagtactacaataagcagtacctggagcgaacgcgggccgagctggacacggcgtgcagacacaactacgaggagacggaggtccccacctccctgcggggtggatcaggtggcgaagacgacattgaggccgaccacgtaggcttctatggtacaactgtttatcagtctcctggagacattggccagtacacacatgaatttgatggtgatgagttgttctatgtggacttggataagaagaaaactgtctggaggcttcctgagtttggccaattgatactctttgagccccaaggtggactgcaaaacatagctgcagaaaaacacaacttgggaatcttgactaagaggtcaaatttcaccccagctaccaatgaggctcctcaagcgactgtgttccccaagtcccctgtgctgctgggtcagcccaacacccttatctgctttgtggacaacatcttcccacctgtgatcaacatcacatggctcagaaatagcaagtcagtcacagacggcgtttatgagaccagcttcctcgtcaaccgtgaccattccttccacaagctgtcttatctcaccttcatcccttctgatgatgacatttatgactgcaaggtggagcactggggcctggaggagccggttctgaaacactgggaacctgagattccagcccccatgtcagagctgacagagactggcggaggtggctcaggcggaggtggatctggaggtggaggctcacggcttgaacagcccaatgtcgccatctccctgtccaggacagaggccctcaaccaccacaacactctggtctgttcggtgacagatttctacccagccaagatcaaagtgcgctggttcaggaatggccaggaggagacagtgggggtctcatccacacagcttattaggaatggggactggaccttccaggtcctggtcatgctggagatgacccctcatcagggagaggtctacacctgccatgtggagcatcccagcctgaagagccccatcactgtggagtggagggcacagtccgagtctgcccggagcaag

SEQ ID NO: 111 - protein, 448 aa
SRLSKVAPVIKARMMEYGTTGGGGSGGGGSGGGGSGDSERHFVHQFKGECYFTNGTQRIRLVTRYIYNREEYLRFDSDVGEYRAVTELGRHSAEYYNKQYLERTRAELDTACRHNYEETEVPTSLRGGSGGEDDIEADHVGFYGTTVYQSPGDIGQYTHEFDGDELFYVDLDKKKTVWRLPEFGQLILFEPQGGLQNIAAEKHNLGILTKRSNFTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWEPEIPAPMSELTETGGGGSGGGGSGGGGSRLEQPNVAISLSRTEALNHHNTLVCSVTDFYPAKIKVRWFRNGQEETVGVSSTQLIRNGDWTFQVLVMLEMTPHQGEVYTCHVEHPSLKSPITVEWRAQSESARSK

SEQ ID NO: 112 - DNA, 318 bp; single-stranded, linear
  CDS 1..318
cggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccacgtggagcatccgagcctgaagagccccatcactgtggagtggagggcacagtctgagtctgcccggagcaag

SEQ ID NO: 113 - protein, 106 aa
RLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESARSK

SEQ ID NO: 114 - DNA, 1323 bp; single-stranded, linear
  CDS 1..1323
ttctttaaaaacatcgtgactccgcgtacacccccgccaggaggtggaggctctggaggtggaggctcaggaggaggtgggtccggagactccgaaaggcatttcgtgttccagttcaagggcgagtgctacttcaccaacgggacgcagcgcatacgatctgtggacagatacatctacaaccgggaggagtacctgcgcttcgacagcgacgtgggcgagtaccgcgcggtgaccgagctggggcggccagaccccgagtactacaataagcagtacctggagcaaacgcgggccgagctggacacggtgtgcagacacaactacgagggggtggagacccacacctccctgcggggtggatcaggtggcgaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaaaacccttatctgctttgtggacaacatcttccctcctgtgatcaacatcacatggctcagaaatagcaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacattgggaacctgagattccagcccccatgtcagagctgacagagactggcggaggtggctcaggcggaggtggatctggaggtggaggctcacggcttgaacagcccaatgtcgtcatctccctgtccaggacagaggccctcaaccaccacaacactctggtctgctcagtgacagatttctacccagccaagatcaaagtgcgctggttccggaatggccaggaggagacggtgggggtctcatccacacagcttattaggaatggggactggaccttccaggtcctggtcatgctggagatgacccctcggcggggagaggtctacacctgccacgtggagcatccgagcctgaagagccccatcactgtggagtggagggcacagtctgagtctgcccggagcaag

SEQ ID NO: 115 - protein, 441 aa
FFKNIVTPRTPPPGGGGSGGGGSGGGGSGDSERHFVFQFKGECYFTNGTQRIRSVDRYIYNREEYLRFDSDVGEYRAVTELGRPDPEYYNKQYLEQTRAELDTVCRHNYEGVETHTSLRGGSGGEDDIEADHVGVYGTTVYQSPGDIGQYTHEFDGDEWFYVDLDKKETIWMLPEFGQLTSFDPQGGLQNIATGKYTLGILTKRSNSTPATNEAPQATVFPKSPVLLGQPKTLICFVDNIFPPVINITWLRNSKSVTDGVYETSFLVNRDHSFHKLSYLTFIPSDDDIYDCKVEHWGLEEPVLKHWEPEIPAPMSELTETGGGGSGGGGSGGGGSRLEQPNVVISLSRTEALNHHNTLVCSVTDFYPAKIKVRWFRNGQEETVGVSSTQLIRNGDWTFQVLVMLEMTPRRGEVYTCHVEHPSLKSPITVEWRAQSESARSK

SEQ ID NO: 116 - DNA, 588 bp; single-stranded, linear
  CDS 1..588
gaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaacacccttatctgctttgtggacaacatcttccctcctgtgatcaacatcacatggctcagaaatagcaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacactgggaacctgagattccagcccccatgtcagagctgacagagact

SEQ ID NO: 117 - protein, 196 aa
EDDIEADHVGVYGTTVYQSPGDIGQYTHEFDGDEWFYVDLDKKETIWMLPEFGQLTSFDPQGGLQNIATGKYTLGILTKRSNSTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWEPEIPAPMSELTET

SEQ ID NO: 118 - DNA, 702 bp; single-stranded, linear
  CDS 1..702
ttctttaaaaacatcgtgactccgcgtacacccccgccaggaggtggaggctctggaggtggaggctcaggaggaggtgggtctggcggtggaggttccggcggaggcggttcagaagacgacattgaggccgaccacgtaggcgtctatggtacaactgtatatcagtctcctggagacattggccagtacacacatgaatttgatggtgatgagtggttctatgtggacttggataagaaggagactatctggatgcttcctgagtttggccaattgacaagctttgacccccaaggtggactgcaaaacatagctacaggaaaatacaccttgggaatcttgactaagaggtcaaattccaccccagctaccaatgaggctcctcaagcgactgtgttccccaagtcccctgtgctgctgggtcagcccaacacccttatctgctttgtggacaacatcttccctcctgtgatcaacatcacatggctcagaaatagcaagtcagtcacagacggcgtttatgagaccagcttccttgtcaaccgtgaccattccttccacaagctgtcttatctcaccttcatcccttctgacgatgatatttatgactgcaaggtggagcactggggcctggaggagccggttctgaaacactgggaacctgagattccagcccccatgtcagagctgacagagact

SEQ ID NO: 119 - protein, 234 aa
FFKNIVTPRTPPPGGGGSGGGGSGGGGSGGGGSGGGGSEDDIEADHVGVYGTTVYQSPGDIGQYTHEFDGDEWFYVDLDKKETIWMLPEFGQLTSFDPQGGLQNIATGKYTLGILTKRSNSTPATNEAPQATVFPKSPVLLGQPNTLICFVDNIFPPVINITWLRNSKSVTDGVYETSFLVNRDHSFHKLSYLTFIPSDDDIYDCKVEHWGLEEPVLKHWEPEIPAPMSELTET

SEQ ID NO: 120 - DNA, 279 bp; single-stranded, linear
  CDS 1..279
ggggacacccgaccacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcgg

SEQ ID NO: 121 - protein, 93 aa
GDTRPRFLWQPKRECHFFNGTERVRFLDRYFYNQEESVRFDSDVGEFRAVTELGRPDAEYWNSQKDILEQARAAVDTYCRHNYGVVESFTVQR